(19) United States
(12) Patent Application Publication
(10) Pub. No.: US 2005/0026866 A1
(43) Pub. Date: Feb. 3, 2005

(54) Agents and methods for treatment of disease by oligosaccharide targeting agents

(76) Inventor: John M. Pawelek, Hamden, CT (US)

Correspondence Address: MILDE & HOFFBERG, LLP;COUNSELORS IN INTELLECTUAL PROPERTY LAW, SUITE 460, 10 BANK STREET, WHITE PLAINS, NY, 10606, US

(21) Appl. No.: 10/631,651

(22) Filed: Jul. 31, 2003

Related U.S. Application Data
(60) Provisional application No. 60/401,183, filed on Aug. 2, 2002.
(60) Provisional application No. 60/451,610, filed on Mar. 3, 2003.

(51) Int. Cl. G01N 33/574; G01N 33/554; G01N 33/569; A61K 31/739

(52) U.S. Cl. 514/54; 435/7.23; 435/7.32

(57) ABSTRACT

A method for targeting, treating, or diagnosing malignant mammalian tumor cells, comprising administering an effective amount of a β1,6-branched oligosaccharide specific binding agent to the mammal. As a treatment, the binding agent may be intrinsically cytotoxic, initiate an endogenous cytotoxic cascade, or play a role in a cytotoxic cascade involving exogenous factors. A preferred binding agent is Bordetella pertussis, which is both specific for the β1,6-branched oligosaccharide and well tolerated. Genetically engineered organisms may also be employed. Pharmaceutical compositions may also serve as binding agents.

### REFERENCE TO EARLIER APPLICATION

The present application claims benefit of priority from U.S. Provisional Application No. 60/401,183, filed Aug. 2, 2002, and 60/451,610, filed Mar. 3, 2003.

### FIELD OF THE INVENTION

The present invention relates to the field of metastatic cell biology, and more particularly to agents and methods for targeting metastatic cells based on particular oligosaccharide properties thereof.

### BACKGROUND OF THE INVENTION

Aberrant glycosylation is a hallmark of malignancy, and includes alterations in the carbohydrate content of glycoproteins, glycolipids, and glycosaminoglycans. A well-studied class are the β1,6-branched oligosaccharides on N-glycans, associated with malignant transformation of rodent and human cells, and poor prognosis in cancer patients. β1,6-N-acetylglucosaminyltransferase V (GNT-V; E.C.2.4.1.155) is a trans-Golgi enzyme that catalyzes the transfer of N-acetylglucoseamine (GlcNAc) from UDP-GlcNAc to β1,6-mannose in the pentasaccharide core of acceptor glycans, forming a β1,6-branched structure in the production of tri- or tetra-antennary N-glycans. β1,6-GlcNAc-linked, polylactosamine antennae on N-glycans are a normal feature of granulocytes and monocytes, and have also been associated with malignant cells. The polylactosamine antennae are carriers of Lewis^{x} and Lewis^{a} antigens, used on N—, and O-glycans by both normal leukocytes and tumor cells in selectin binding during intravasation and systemic migration.

Elevated GNT-V expression has been shown to result in loss of contact inhibition and decreased substrate adhesion, increased susceptibility to apoptosis, and increased tumorigenicity in nude mice. GNT-V-deficient mice showed suppressed tumor growth and lowered incidence of metastasis. Increased β1,6-branched N-glycans on β1 integrin reduced α₅β₁ integrin clustering and stimulated in vitro migration of human fibrosarcoma cells.

The lectin, leukocytic phytohemagglutinin (LPHA, phaseola vulgaris), shows high-affinity binding to β1,6-branched oligosaccharides, and can be used in lectin histochemistry to detect these oligosaccharides in formalin-fixed, paraffin-embedded tissues. However, little has been reported on the histology of LPHA-positive cells in human cancer, and the two existent studies investigated only primary tumors. One study of primary breast and colon carcinomas described LPHA staining of “coarse granules and globules located in the cytoplasm in a supranuclear position” (Fernandes B., Sagman U., Auger M., Demetrio M., Dennis J. W. β1,6-branched oligosaccharides as a marker of tumor progression in human breast and colon neoplasia. Cancer Res. 51: 718-723, 1991.). Another study of primary breast carcinomas described LPHA reactivity as “diffuse cytoplasmic staining, sometimes concentrated in the Golgi area, or at the plasma membrane.” (Chammas, R., Cella, N., Marques, L. A., Brentani, R. R., Hynbes, N. E., and Franco, E. L. F. re: B. Fernandes et al., beta 1-6 branched oligosaccharides as a marker of tumor progression in human breast and colon neoplasia. Cancer Res., 51: 718-723, 1991. [letter; comment.]. Cancer Res. 54: 306-307, 1994.). These studies gave no indication as to the possible degree of LPHA positivity, if any, in metastatic tumors.

A major problem in the chemotherapy of solid tumor cancers is the delivery of therapeutic agents, such as drugs, in sufficient concentrations to eradicate tumor cells while at the same time minimizing damage to normal cells. Thus, studies in many laboratories are directed toward the design of biological delivery systems, such as antibodies, cytokines, and viruses for targeted delivery of drugs, pro-drug converting enzymes, and/or genes into tumor cells. Houghton and Colt, 1993, New Perspectives in Cancer Diagnosis and Management 1: 65-70; de Palazzo, et al., 1992a, Cell. Immunol. 142:338-347; de Palazzo et al., 1992b, Cancer Res. 52: 5713-5719; Weiner, et al., 1993a, J. Immunotherapy 13:110-116; Weiner et al., 1993b, J. Immunol. 151:2877-2886; Adams et al., 1993, Cancer Res. 53:4026-4034; Fanger et al., 1990, FASEB J. 4:2846-2849; Fanger et al., 1991, Immunol. Today 12:51-54; Segal, et al., 1991, Ann N.Y. Acad. Sci. 636:288-294; Segal et al., 1992, Immunobiology 185:390-402; Wunderlich et al., 1992; Intl. J. Clin. Lab. Res. 22:17-20; George et al., 1994, J. Immunol. 152:1802-1811; Huston et al., 1993, Intl. Rev. Immunol. 10:195-217; Stafford et al., 1993, Cancer Res. 53:4026-4034; Haber et al., 1992, Ann. N.Y. Acad. Sci. 667:365-381; Haber, 1992, Ann. N.Y. Acad. Sci. 667: 365-381; Feloner and Rhodes, 1991, Nature 349:351-352; Sarver and Rossi, 1993, AIDS Research & Human Retroviruses 9:483-487; Levine and Friedmann, 1993, Am. J. Dis. Child 147:1167-1176; Friedmann, 1993, Mol. Genetic Med. 3:1-32; Gilboa and Smith, 1994, Trends in Genetics 10:139-144; Saito et al., 1994, Cancer Res. 54:3516-3520; Li et al., 1994, Blood 83:3403-3408; Vieweg et al., 1994, Cancer Res. 54:1760-1765; Lin et al., 1994, Science 265:666-669; Lu et al., 1994, Human Gene Therapy 5:203-208; Gansbacher et al., 1992, Blood 80:2817-2825; Gastl et al., 1992, Cancer Res. 52:6229-6236.

Because of their biospecificity, biological delivery systems could in theory deliver therapeutic agents to tumors. However, it has become apparent that numerous barriers exist in the delivery of therapeutic agents to solid tumors that may compromise the effectiveness of antibodies, cytokines, and viruses as delivery systems. Jain, 1994, Scientific American 7:58-65. For example, in order for chemotherapeutic agents to eradicate metastatic tumor cells, they must a) travel to the tumors via the vasculature; b) extravasate from the small blood vessels supplying the tumor; c) traverse through the tumor matrix to reach those tumor cells distal to the blood supply; and d) interact effectively with the target tumor cells (adherence, invasion, pro-drug activation, etc).

Live bacteria were first deliberately used in the treatment of cancer nearly 150 years ago, work that ultimately led to the field of immunomodulation. Today, with the discovery of bacterial strains that specifically target tumors, and aided by the advent of genomic sequencing and genetic engineering, there is new interest in the use of bacteria as tumor vectors. Bifodobacterium, Clostridium, and Salmonella have all been shown to preferentially replicate within solid tumors compared to normal tissues when injected from a distal site, and all three bacteria have been genetically engineered as tumor vectors, to transport and amplify genes encoding factors such as prodrug-converting enzymes, toxins, angiogenesis inhibitors, and immune-enhancing cytokines. The purpose of this article is to provide an historical review of this field, and to focus on the current development of these bacteria as they are today being readied for clinical trials in cancer patients.

Perhaps the first cancer patient to be purposefully infected with bacteria was treated by German physician W. Busch (1). Busch, in 1868, induced a bacterial infection in a woman with inoperable sarcoma by cauterizing the tumor and placing her into bedding previously occupied by a patient with ‘erysipelas’ (Streptococcus pyrogenes). Busch reported that within a week the primary tumor had shrunk by half and that lymph nodes in the neck had also shrunk in size, however, the patient collapsed and died nine days after the infection had begun (1). Almost 30 years later, William B. Coley, a young surgeon at New York Hospital, encountered a cancer patient who seemed to be cured by a severe infection with erysipelas (2-3). Coley wrote, “I had found one case of very malignant round-celled sarcoma of the neck, four times recurrent, in which an attack of erysipelas had accidentally occurred shortly after the last operation by Dr. Bull. At this time the tumor so extensively involved the deeper tissues of the neck that no attempt was made to remove it. A few days after the first attack of erysipelas had subsided, a second attack followed, lasting for a week. During these attacks of erysipelas, the tumor of the neck entirely disappeared and the patient left the hospital in good health. After great effort I finally succeeded in tracking the after-history of this patient and found him alive and well in 1891, seven years later.” This observation led Coley to begin deliberate infection of cancer patients with live S. pyrogenes. Unbeknownst to Coley, similar studies had already been launched in Europe, where, in 1883, Friedrich Fehleisen, a German surgeon, had not only successfully identified S. pyrogenes as the cause of erysipelas, but had at once begun treating cancer patients with the living cultures of the bacteria (4).

Both Coley and Fehleisen reported success in eliciting tumor regression, and Coley was so convinced by his results that he devoted much of his life's work to exploring the use of bacteria in cancer treatment. Coley soon abandoned the use of live bacteria in favor of isolated preparations of bacterial toxins. A record of his work was carefully assembled by his daughter, Helen Coley Nauts, which also summarized case reports over 200 years wherein neoplasms regressed following acute infection (5-6). It seemed that when neither the cancer nor the infection was too far-advanced, yet the infection was of sufficient severity or duration, some tumors completely disappeared and the patients remained free from recurrence. However, these studies were controversial, because they were anecdotal and difficult to repeat, and would not live up to current standards for such clinical trials. Nonetheless, subsequent evidence in mouse tumor models indicated that at least some of the anti-cancer effects of bacterial infections might have indeed reduced tumor size, and, in part, the effects seemed to have been mediated through stimulation of the host immune system. Carswell et al. (7) first reported that endotoxin (lipopolysaccharide, LPS) from gram-negative bacteria triggers release of tumor necrosis factor alpha (TNFα), by cells of the immune system, initiating a cascade of cytokine-mediated reactions, culminating in the destruction of tumor cells (8). Subseqently, bacterial adjuvants were shown to be immuno-enhancing in cancer patients (e.g. 9-11). Today, these and numerous related studies have culminated in the large and diverse field of cancer immunotherapy, of which William Coley is generally recognized as the founder (1). In addition, the use of bacterial toxins in cancer therapy remains a topic of considerable current interest (12).

Clostridum

In the early studies above, there was no concept of using live bacteria as vectors, i.e. organisms that preferentially populate tumors following distal inoculation into the circulatory system, carrying inherent or engineered anticancer agents to the tumor. It was several decades after the work of Fehleisen and Coley before the first attempts to employ bacteria as tumor vectors were carried out, initially with spores of the Clostridum family. Clostridia are a group of anaerobes, and their successful colonization of necrotic tissue is common, resulting in gas gangrene. As early as 1813, Vautier reported cancer patients that appeared to be cured when the patient acquired gas gangrene (1). In 1947, it was first shown that direct injection of spores of Clostridium histolyticus into a transplantable mouse sarcoma caused oncolysis (liquification) and tumor regression (13). However, very few animals survived this treatment, as Clostridium-mediated oncolysis was accompanied by acute toxicity and death of the mice, a phenomenon subsequently documented by several laboratories (14-19). Möse and Möse (15) using a ‘non-pathogenic’ soil isolate Clostridium butyricum, strain M-55, described the colonization and oncolysis of Erlich ascites tumors following i.v. injections of bacteria as follows, “A few days after the injection of the spores, the tumor softened noticeably and shortly thereafter fluctuated on palpation. At this time it usually broke through to the outside with spontaneous discharge of brownish liquid necrotic masses, which had the consistency of thin pus. The afflicted leg frequently died off, and a large cavity remained which sometimes reached close to the peritoneum. The animals usually did not survive this stage of oncolysis for any length of time. At this point, the tumor appeared macroscopically to have disappeared completely; nevertheless histologically in many cases one could find at the inner edge of the cavity more or less abundant tumor tissue that was covered by a layer of necrotic material. Permanent survival of the animals occurred only rarely and then only if the tissue defect had not been too large.” These effects of the Clostridia spores were apparently due to their ability to germinate within necrotic, anaerobic areas of tumors. Not all spore forming bacteria were effective, as facultatively anaerobic spore-forming organisms, Bacillus mesentericus or Bacillus subtilis, which were prepared in a similar manner, did not elicit oncolysis (however, tumor-targeting was not assessed). This indicated that although the anerobic phenotype of Clostridium is the probable underlying basis for their specific targeting of necrotic areas of tumor (15), other factors may be involved in their ability to grow there. However, clostridial spores only achieved germination and colonization when the tumors were large enough for significant anoxia. In a metastatic mouse tumor model, following i.v.-injection of spores of strain M-55, metastases in organs or lymph nodes were unaffected by the spores unless the metastatic tumors had reached a considerable size (2-4 g) (18). Likewise, i.v. injected spores of a number of species of nonpathogenic Clostridia, including M-55, produced no effect when administered when tumors were small. As described by Thiele et al (17), “The qualitative differences in germination of spores were likely to be not a characteristic of neoplastic and normal tissues per se, but related to physiologic and biochemical conditions found within large tumor masses. Thus, Clostridial oncolysis could not be expected to be successful in seeking out small clusters of tumor cells which enjoy good circulation and nutrition.”

Though tumor size limitations remain a characteristic of Clostridiums strains currently employed, strains are now available with greatly reduced toxicity, and thus prolonged survival time can be achieved by Clostridium injections into tumor-bearing animals. In initial studies, Fox et al., using a Clostridium expression vector, were able to transform the E. coli cytosine deaminase gene into Clostridium beijerincki, resulting in increased cytosine deaminase activity in extracts of the transformed clostridial bacteria (20). These extracts, when added to cultures of mouse EMT6 carcinoma cells, sensitized the cells to 5-fluorocytosine, through its conversion to the toxic 5-fluorouracil via E. coli cytosine deaminase (20). Similarly, Minton et al. inserted the E. coli nitroreductase gene into Clostridium beijerincki and were able to detect expression of this gene in an in vivo murine tumor model through the use of antibodies directed against the E. coli nitroreductase gene (21). Nitroreductase activates CB1954, a potent alkylating agent. Recent studies of Clostridia as tumor vectors have focused on their potential in gene therapy and controlled gene expression through use of radio-inducible promoters in vivo (22-26). Another group has used Clostridium in combination with chemotherapy (27), demonstrating significantly improved antitumor activity compared to either the bacteria or the chemotherapy alone. Thus, many years after the first injection of Clostridium spores into tumors, a number of recent advances have now demonstrated good promise for Clostrium as a tumor-targeting, therapeutic vector.

Bifidobacterium

As in the case of Clostridia, Bifididobacterium is a group of gram-positive anaerobic bacteria that have been found to colonize large tumors, very likely because of the requirement for the anaerobic growth environment present in parts of large tumors. In contrast to Clostridium, however, Bifidobacteria are non-pathogenic, non-spore-forming and found naturally in the digestive tract of humans and certain other mammals, and they thus have the potential of being safer to use as a live bacterial agents in treating tumors. Cell wall extracts have been used as immuno-modulators, similar to BCG (28-29). In the first of these tumor-targeting studies, Kimura et al., (30) used Ehrlich ascites tumors implanted in the thigh muscle of DDD-H-2^{s} mice. A suspension of lyophilized Bifidobacteria was injected in the tail vein. Proliferation and/or survival of the bacteria was assisted by daily intraperitoneal injection of lactulose. By adding lactulose, a sugar substrate for the bacteria that is not metabolized by mammalian cells, the relative growth and survival of the bacteria increased within the tumor by 1000 fold compared to a saline control. Targeting of the bacteria showed highly specific tumor localization, with virtually no bacteria in other organs after 96 hours. Bacteria within the tumor at 1 hour were present at 10²/g and rose to 10⁶/g by day 7. With an injection of 5×10⁶ c.f.u.per mouse, tumor targeting occurred best with tumors 1.5 cm in diameter or greater. At the same dose, targeting to tumors smaller than 1.5 cm resulted in a significant drop in the percentage of tumors targeted. Higher doses allowed colonization of increased numbers of smaller tumors. No antitumor effect or prolongation of survival was found in these studies. Subsequent work showed that the Bifidobacterium also targets carcinogen-induced mammary tumors in rats, believed to be more representative of naturally occurring tumors (31).

Evidence that Bifidobacterium could deliver effector genes to tumors was provided by Yazawa et al., using B. lougum, (32). The ability of the bacteria to carry a plasmid bearing a spectinomycin-resistance marker to B16-F10 melanomas or Lewis lung carcinomas in mice was assessed, and spectinomycin-resistant colonies were obtained for both tumor types. Likewise, B. adolescentis, engineered with a plasmid encoding the endostatin gene, was shown to target the Heps mouse liver tumor implanted s.c. in BALB/c mice, and to inhibit both angiogenesis and growth of the tumor (33). Together, these data demonstrate that Bifidobacterium can be used to deliver a plasmid-encoded antitumor effector genes and thus joins the growing list of live bacteria as potential tumor-targeting anticancer vectors.

Salmonella

Salmonella are gram-negative, facultative anaerobes that are a frequent cause of intestinal infections. Salmonella are also known to inherently colonize human tumors (34-37). Because of the high-level immunostimulation of Salmonella LPS and other components, systemic infections with Salmonella induces septic shock and high mortality in humans if not treated soon enough. However, early studies by Bacon et al. demonstrated that Salmonella virulence in mice was attenuated in certain auxotrophic mutants (38-40). In 1997, it was first reported that Salmonella auxotrophs, when injected into tumor-bearing mice, would preferentially replicate within the tumors, achieving tumor to normal tissue ratios often exceeding 1000:1 (41). Because Salmonella grow under both aerobic and anaerobic conditions, they are able to colonize both large and small tumors. Salmonella have also been shown to inhibit a melanoma metastasis model causing a considerable reduction in the size and number of micrometastasis (42).

A surprising finding was the ability of attenuated Salmonella to retard tumor growth in a broad range of human and mouse tumors implanted in mice. In most cases tumor growth was inhibited for prolonged periods, in some cases several weeks after untreated, tumor-bearing mice had succumbed. These observations, coupled with the ease of genetic manipulation, suggested that Salmonella were good candidates as therapeutic anticancer agents, and accordingly, genetically engineered Salmonella were developed to express effector genes such as those encoding the herpes simplex thymidine kinase (41, 43-47), E. coli cytosine deaminase (48), tumor necrosis factor alpha (TNFα) (49), and colicin E3 (50). See also, U.S. Pat. No. 6,190,657, expressly incorporated herein by reference in its entirety.

To reduce the possibility of LPS-induced septic shock in cancer patients treated with Salmonella, lipid A-modified (msbB) Salmonella auxotrophs (purI⁻) were developed that were attenuated for toxicity in mice and swine (43). These mutants showed significantly reduced host TNFα induction, yet retained the abilities for tumor-targeting, amplification, and growth suppression in mice, achieving tumor accumulations of 10⁹-10¹⁰ colony forming units (cfu)/g tumor with tumor to normal tissue ratios exceeding 1000:1. Below a number of experiments are presented illustrating the potential of Salmonella as a tumor-targeting vector.

Salmonella Tumor Colonization Viewed by Electron Microscopy

Salmonella infection of mouse melanomas and a diverse array of human carcinomas implanted in mice was studied by electron microscopy. Bacteria were injected i.v. into tumor-bearing mice five days prior to sacrifice. As was the case with all the tumors analyzed, including a diverse may of human carcinomas, the vast majority of Salmonella were seen in necrotic regions. However in some cases, bacteria were visible in the melanoma cell cytoplasm, in this case along with numerous melanosomes. To investigate the what mechanisms by which Salmonella achieve tumor infection and amplification following i.v. or i.p. injection, the potential roles of two major pathogenicity islands on the Salmonella chromosome, SPI-1 and SPI-2, known to be involved with growth and survival of Salmonella during systemic infection of the host, were studied.

Salmonella Pathogenicity Islands and the Anticancer Phenotype

The intratumoral environment is highly complex, presenting not only diverse physico-chemical barriers, but also tumor-infiltrating leukocytes comprised of macrophages, dendritic cells, lymphocytes, and neutrophils with antimicrobial properties. The ability of Salmonella to survive and amplify in the midst of these barriers is key to its use as an anticancer vector. Salmonella enterica servovar Typhimurium contains about 200 genes for virulence factors, encoded on five pathogenicity islands, smaller pathogenicity islets, at least one virulence plasmid, and other chromosomal sites (51-54). There are also at least two type III secretion systems (TTSS). One (Inv/Spa) is located in SPI-1 and controls bacterial invasion of epithelial cells during dissemination from the gut (55-56). The other is located in SPI-2 where it plays a crucial role in systemic growth of Salmonella in its host and is required for survival within macrophages and epithelial cells (57-61). Through analyses of disabling mutations, we concluded that expression of SPI-2, but not SPI-1, is essential for the Salmonella antitumor effects, at least in part by aiding bacterial targeting of, and amplification within tumors (62). Disabling SPI-1 (prgH⁻) reduced invasion in vitro by 100 fold, but had no effect on tumor growth suppression in vivo. However, disabling SPI-2 (ssaT⁻) ablated tumor growth suppression. In addition to ssaT⁻ Salmonella, derivatives in translocon (and putative effector) genes sseA, sseB, sseC, putative chaperone gene sscA, or regulatory gene ssrA were unable to delay tumor growth, while mutants in effector genes sseF and sseG yielded partial growth delay compared the SPI-2⁺ counterpart. Impaired tumor amplification was seen in SPI-2 mutants after either intervenous or intratumoral injection. A SPI-2⁻ strain was unable to suppress tumor growth in CD18-deficient mice with defective macrophages and neutrophils, suggesting that loss of tumor growth suppression in wild type mice by SPI-2 mutants was not solely a function of increased susceptibility to immune attack. Thus SPI-2 is essential for the Salmonella antitumor effects, perhaps by aiding bacterial amplification within tumors, and is the first identified genetic system for this Salmonella phenotype. However the mechanisms remain unknown, and further studies are necessary to understand these highly complex molecular pathways through which the Salmonella anticancer phenotype is achieved.

Development of Safe Vectors with Altered Lipid A (msbB⁻)

The concept of systemically administering gram-negative wild type bacteria such as Salmonella into humans raises the crucial issue of the natural ability of these bacteria to induce septic shock mediated by tumor necrosis factor TNFα(63-64). However, studies of lipid biosynthesis have shown that in Escherichia coli and Salmonella certain genetic blocks greatly lower TNFα induction and render the bacteria substantially non-toxic. In particular in E. coli, genetic disruption of the msbB gene, needed for the terminal myristoylation of lipid A (65-66), results in a stable non-conditional mutation which lowers TNFα induction up to 10-fold by whole bacteria or up to 10,000-fold by purified LPS. A similar toxicity profile was reported when msbB was disrupted in Salmonella (67). We generated a deletion in the coding sequence of msbB within one of our hyperinvasive Salmonella strains previously used for tumor-targeting (43). We found that in Salmonella, the msbB⁻ mutation results in phenotypic growth defects, under certain conditions in vitro, in contrast to E. coli where this is not observed. In Salmonella msbB⁻ strains, secondary mutations that partially suppress the MsbB⁻ phenotype and produce fitter strains arise with high frequency (68).

These live msbB⁻ bacteria (with and without growth suppressors), and their isolated lipids were indeed found to have a reduced ability to elicit TNFα in animals. In mice, live Salmonella wild type and msbB⁻ strains were compared for TNFα induction 1.5 hr after infection. TNFα induction was only 33% of wild typein mice treated with the msbB⁻ strains (Table 1). Similarly, live msbB⁻ bacteria were also found to have a reduced ability to elicit TNFα in Sinclair swine. Bacteria lacking msbB, injected into the ear vein of Sinclair swine, induced TNFα at 14% of the amount induced by wild type.

This reduction in TNFα induction was accompanied by a striking reduction in virulence in vivo. Whereas as few as 20 cfu of i.p. injected wild type Salmonella caused death in mice, injection of 2×10⁷ cfu msbB⁻ Salmonella resulted in negligible mortality, allowing 100% of the mice to survive past 28 days. Similarly, when 10⁹ cfu were injected into the ear veins of Sinclair swine, wild type Salmonella killed 90% of the swine in 5 days, whereas the same number of msbB⁻ mutant cells allowed 100% of the swine to survive past 28 days.

In addition, even with the reductions in TNFα and increased attenuation in vivo, these msbB⁻ bacteria retained the ability to target tumors and retard their growth. Tumor-targeting and colonization were first tested using the B16F10 melanoma implanted s.c. in C57B6 mice. Five days after administration of 10⁵ cfu bacteria, tumor levels ranged from 10⁸-10⁹ per g of tumor, exhibiting a positive targeting ratio between 1000:1 and 2000:1 as compared to the liver (Table 2). The presence of the msbB⁻ mutation in the Salmonella also did not diminish the tumor inhibition activity against subcutaneously implanted B16F10 melanoma. Similar to attenuated but msbB⁺ tumor-targeting strains (41), msbB⁻ strains showed highly significant inhibition of tumor growth. For example, at day 18 the T/C % inhibition of strain YS8211 was 94%, and strain YS1629 was 96%.

Thus, msbB⁻ Salmonella appear to be good candidates as safe vectors. Indeed, one msbB⁻ strain, VNP20009 (45), is currently in Phase I clinical trials, with the first report of one trial demonstrating a maximim tolerated dose of 3×10⁸cfu/m², and tumor targeting in some patients (69-70). That these bacteria can be used safely in humans has encouraged further development to genetically engineer strains to produce foreign proteins with anticancer activities, as described below.

Tumor Amplified Protein Expression Therapy (TAPET™)

The potential for bacteria to serve as protein expression systems is enormous. Salmonella and other bacteria which target tumors extend this potential to include both the delivery and expression of anticancer therapeutic proteins directly within cancerous tissue. While bacteria do not perform mammalian glycosylation and other protein modifications, there are many effector proteins in which such modifications are unnecessary. The herpes simplex thymidine kinase (HSV TK) is an example of a prodrug-converting enzyme that is functionally expressed in bacteria (41, 46, 71). This enzyme activates nucleoside analogues such as acyclovir (ACV) and ganciclovir (GCV). We used a strain of Salmonella expressing a secreted form of HSV TK in a B16F10 subcutaneous melanoma model, and observd that 1) the presence of the plasmid vector alone lessened the innate antitumor activity of the bacteria, and 2) when these bacteria were co-administered with GCV the resulting tumors were 2.5 times smaller than without added GCV. This study indicated that these bacteria were capable in delivering a prodrug-converting enzyme effective in activating a compound into its chemotherapeutic form.

Diagnostic Imaging of Tumors

The diagnostic imaging of tumors is yet another potentially powerful application of tumor targeting, and the HSV-TK system was shown to be a useful model for this approach. Localization of [¹⁴C]-2′-fluoro-2′-deoxy-5-iodouracil-β-D-arabinofuranoside (FIAU) in tumored mice pretreated with Salmonella expressing HSV-TK was demonstrated (72). The [¹⁴C]-FIAU radioactivity and bacterial count data showed a Salmonella^{TK}-dependent [¹⁴C]-FIAU accumulation of at least 30-fold higher in tumor tissue compared to muscle tissue. These results provided direct proof of intratumoral prodrug conversion, and further demonstrated the feasibility of Salmonella-mediated delivery of diagnostic imaging markers.

Antitumor Effects of Salmonella in Combination with Radiation

The combination of two modes of cancer therapy which differ in their therapeutic targets has often improved the resulting therapeutic index. Thus, it was investigated whether Salmonella might be useful when combined with X-ray therapy for melanomas and other solid tumors (73). Recent studies have shown that X-ray treatment of melanomas can elicit local control and even complete responses in a significant percentage of patients (74). Therefore there was good reason to test the effectiveness of combined treatments of Salmonella+X-rays against melanomas and other solid tumors. The effects of single X-ray doses ranging from 5 to 15 Gy on B16F10 growth suppression with and without Salmonella (injected i.v.) were determined. Anti-tumor activity was measured as the number of days post tumor implantation needed to form 1 g tumors. X-rays alone (open circles) prolonged the time to 1 g in a dose-dependent fashion. Salmonella alone (closed circles, 0Gy) prolonged the time to 1 g from the control value (open circles, 0Gy) of 18+1 d to a value of 26+3d. Surprisingly, the combination of Salmonella+X-rays showed supra-additive anti-tumor effects, with the slope of the dose-response curve being greater than expected for additivity. Supra-additivity was indicated in all 3 of the 3 X-ray dose-response experiments in mice using the B16F10 melanoma, as shown by comparing the actual slopes of the dose-response curves obtained to those slopes expected for simple additivity. Tumor growth curves show that the combination of Salmonella and a singe dose of 15Gy X-rays markedly slowed B16F10 melanoma growth and prolonged mouse survival compared to the other treatment catagories. Similar results with a single dose of 15Gy X-rays in combination with Salmonella were obtained with the Cloudman S91 melanoma line implanted s.c. in DBA/2J mice. The observation of supra-additivity of the two modes of treatment suggests that they target different sub-populations of tumor cells.

Intratumoral Induction of Reporter Genes by Externally-Applied Stimuli

Externally sustained or pulsed regulation of anticancer genes within tumors could improve the antitumor capabilities of the genes in question. For studies on intratumoral gene induction in Salmonella, we explored two promoter/reporter systems: the luciferase gene controlled by the tetracycline-sensitive promoter (C. Clairmont, J. Pike, K. Troy and D. Bermudes, unpublished), and the colicin E3 gene controlled by an SOS-sensitive promoter (50). Salmonella bearing the luciferase gene fused to a tetracycline-sensitive promoter produced luciferase following i.v. administration of anhydrotetracycline to the mice. Intratumoral luciferase activity was induced by anhydrotetracycline in both the 5 h and 15 h treatment protocols (p vs control <0.010). Likewise, when mouse B16F10 melanomas growing in C57B6 mice were colonized with Salmonella bearing an SOS-inducible colicin E3 gene, they produced intratumoral colicin E3 following i.p. injections of mitomycin C, or externally-applied X-rays. Colicine E3 in tumor supernatants was assayed by its inhibition of growth of E. coli strain MG1655 in Luria broth. There was an 8-10 fold increase in intratumoral colicin E3 activity comparing mitomycin C treated to untreated controls.

Thus, intratumoral activation of two different promoter/reporter gene systems using Salmonella-infected tumors was accomplished. In additional studies not shown, introduction of a recN⁻ mutation increased the bacterial sensitivity to intratumoral SOS-induction of colicin E3 (unpublished data from our labs). These studies demonstrate that regulation of anticancer genes through externally-applied stimuli using genetically engineered Salmonella is a feasible therapeutic approach.

Anecdotal case reports dating back more than 200 years describe tumor regression in patients with severe bacterial infections, and application of bacteria in cancer therapy was pioneered independently by Drs. Friedrich Fehleisen and William B. Coley in the late 1800's and early 1900's, leading eventually to the field of immunomodulation for the treatment of cancer (1-6). A number of more current studies have now demonstrated the potential of genetically-engineered live bacteria as tumor-targeting vectors in human cancer therapy. In animal tumor models, these bacteria target and multiply selectively within tumors, thus amplifying intratumoral gene delivery and therapeutic effects. In some cases the microorganisms also exhibit inherent tumor-suppressing activities; and, in the case of Salmonella, this effect remains even in strains with altered LPS and reduced TNFα induction. This work is greatly advanced by progress in genetic engineering and genomic sequencing.

### REFERENCES

1. Hall, S. S. A Commotion in the Blood, Henry Holt and Company, New York, 544 pp., 1998.
2. Coley, W. B. Contribution to the knowledge of sarcoma. An. Surgery 1891; 14: 199-220.
3. Coley, W. B. Late results of the treatment of inoperable sarcoma by the mixed toxins of erysipelas and Bacillus prodigiosus, Am J Med Sci 1906; 131: 375-430.
4. Fehleisen, F. Die Etiologie des Erysipels. Berlin, 1883. (see ref 1, p. 48).
5. Nauts, H. C., Swift, W. E., Coley, B. L. The treatment of malignant tumors by bacterial toxins as developed by the late William B. Coley, M. D., reviewed in the light of modern research. Cancer Res 1946; 6: 205-216.
6. Nauts, H. C., Fowler, G. A. A., Bogatko, F. H. A review of the influence of bacterial infection and of bacterial products (Coley's toxins) on malignant tumors in man. Acta Medica Scandinavica 1953; 145 (Suppl. 276:1-105).
7. Carswell, E. A., Old, L. J., Kassel R. L., Green, S., Fiore, N., and Williamson, B. An endotoxin induced serum factor that causes necrosis of tumors. Proc Nat Acad Sci USA 1975; 72: 3666-3670.
8. Berg, A. A., and Baltimore, D. An essential role for NF-κB in preventing TNFα-induced death. Science 1996; 274: 782-784.
9. Zbar, B., Bernstein, I., Tanaka, T., and Rapp, H. J. Tumor immunity produced by the intradermal inoculation of living tumor cells and living Mycobacterium bovix (strain BCG). Science 1970; 17:1217-1218.
10. Goonewardene, M. Clinical results and immunologic effects of a mixed bacterial vaccine in cancer patients. Med Oncol Tumor Parmacother 1993; 10: 145-158.
11. Axelrod, R. S., Havas, H. F., Murasko, D. M., Bushnell, B., and Guan, C. F. Effect of the mixed bacterial vaccine on the immune response of patients with non-small cell lung cancer and refractory malignancies. Cancer 1988; 61: 2219-2230.
12. Liu, S., Aaronson, H., Mitola, D. J., Leppla, S. H., and Bugge, T. H. Potent antitumor activity of a urokinase-activated engineered anthrax toxin. Proc Natl Acad Sci USA 2003; 100: 657-62.
13. Parker, R. C., Plummber, H. C., Siebenmann, C. O., and Chapman, M. G. Effect of histolyticus infection and toxin on transplantable mouse tumors. Proc Soc Exp Biol Med 1947, 66: 461-465.
14. Gericke, D., and Engelbart, K. Oncolysis by clostridia. II experiments on a tumor spectrum with a variety of clostridia in combination with heavy metal. Cancer Res 1964; 24: 217-221.
15. Möse, J. R., and Möse, G., Oncolysis by clostridia. I. Activity of Clostridium butyricum (M-55) and other nonpathogenic clostridia against the Ehrlich carcinoma. Cancer Res 1964; 24: 212-216.
16. Thiele E. H., Arison, R. N., and Boxer, G. E. Oncolysis by clostridia. III. Effects of clostridia and chemotherapeutic agents on rodent tumors. Cancer Res 1964; 24: 222-233.
17. Thiele E. H., Arison, R. N., and Boxer, G. E. Oncolysis by clostridia. IV. Effect of nonpathogenic clostridia spores in normal and pathological tissues. Cancer Res 1964; 24: 234-238.
18. Engelbart, K. and Gericke, D. Oncolysis by clostridia. V. Transplanted tumors of the hamster. Cancer Res 1964; 24; 239-243.
19. Carey, W. W., Holland, J. F., Whang, H. Y., Neter, E., Bryant, B. Clostridial oncolysis in man. Eur J Cancer 1967; 3: 37-46.
20. Fox, M. E., Lemmon, M. J., Mauchline, M. L., Davis, T. O., Giaccia A. J., Minton, N. P., Brown, J. M. Anaerobic bacteria as a delivery system for cancer gene therapy: in vitro activation of 5-fluorocytosine by genetically engineered clostridia. Gene Therapy 1996; 3:173-178.
21. Minton, N. P., Mauchline, M. L., Lemmon, M. J., Brehm, J. K., Fox, M., Michael, N. P., Giaccia, A., Brown, J. M. Chemotherapeutic tumor targeting using clostridial spores. FEMS Microbiol Rev 1995; 17: 357-364.
22. Lemmon M J, van Zijl P, Fox Me., Mauchline M L, Giaccia A J, Minton N P, Brown J M. Anaerobic bacteria as a gene delivery system that is controlled by the tumor microenvironment Gene Therapy 1997; 8: 791-796.
23. Theys, J., Landuyt, W., Nuyts, S., Van Mellaert, L., van Oosterom, A., Lambin, P., and Anne, J., Specific targeting of cytosine deaminase to solid tumors by engineered Clostridium acetobutylicum. Cancer Gene Therapy 2001; 8: 294-297.
24. Nuyts, S., Mellaert, I. V., Theys, J., Landuyt, W., Lambin, P., and Anne, J. Clostridium spores for tumor-specific drug delivery. Anti-Cancer Drugs 2002; 13: 115-125s.
25. Liu, S.-C., Minton, N. P., Giaccia, A. J., and Brown, J. M. Anticancer efficacy of systemically delivered anaerobic bacteria as gene therapy vectors targeting tumor hypoxia/necrosis. Gene Therapy 2002; 9: 291-296.
26. Nuyts, S., Van Mellaert, L., Theys, J., Landuyt, W., Bosmans, E., Anne, J., and Lambin, P. Radio-responsive recA promoter significantly increases TNFα production in recombinant clostridia after 2 Gy irradiation. Gene Therapy 2002; 8: 1197-1201.
27. Dang, L. H., Bettegowda, C., Huso, D. L., Kinzler, K. W., and Vogelstein, B. Combination bacteriolytic therapy for the treatment of experimental tumors. Proc Natl Acad Sci USA 2001; 98:15155-15160.
28. Sekine, K., Ohta, J., Onishi, M., Tatsuki, T., Shimokawa, Y., Toida, T., Kawashima, T., Hashimoto, Y. Analysis of antitumor properties of effector cells stimulated with a cell wall preparation (WPG) of Bifidobacterium infantis. Biol Pharm Bull 1995; 18: 148-153.
29. Rhee, Y. K., Bae, E. A., Kim, S. Y., Han, M. J., Choi, E. C., Kim, D. H. Antitumor activity of Bifidobacterium spp. isolated from a healthy Korean. Arch Pharm Res. 2000; 23: 482-487.
30. Kimura, N. T., Taniguchi, S., Aoki, K., Baba, T. Selective localization and growth of Bifidobacterium bifidum in mouse tumors following intravenous administration. Cancer Res 1980; 40: 2061-2068.
31. Yazawa, K., Fujimori, M., Nakamura, T., Sasaki, T., Amano, J., Kano, Y., and Taniguchi, S. Bifodobacterium longum as a delivery system for cancer gene therapy of chemically induced rat mammary tumors. Breast Cancer Research and Treatment 2001; 66: 165-170.
32. Yazawa, K., Fujimori, M., Amano, J., Kano, Y., and Taniguchi, S. Bifodobacterium longum as a delivery system for cancer gene therapy: Selective localization and growth in hypoxic tumors. Cancer Gene Therapy 2000; 7: 269-274.
33. Li, X., Fu, G.-F., Fan, Y.-R., Liu, W.-H., Jiu, X.-J., Wang, J.-J., and Xu, G.-X. Bifidobacterium adolescentis as a delivery system of endostatin for cancer gene therapy: Selective inhibitor of angiogenesis and hypoxic tumor growth. Cancer Gene Therapy 2003; 10: 105-111.
34. Graham F. O., and Coleman P. N. Infection of a secondary carcinoma by Salmonella montevideo. Brit Med J 1952; 1:1116.
35. Giel, C. P. Abscess formation in a pheochromocytoma. NE J Med 1954; 251:980-982.
36. Gill, G. V. and Holden A. A malignant pleural effusion infected with Salmonella enteritidis. Thorax 1996; 51: 104-105.
37. Johnson, P. H., and Macfarlane J. T. Commentary: Pleural empyema and malignancy-Another direction. Thorax 1996; 51:107-108.
38. Bacon G A, Burrows T W, Yates M. The effects of biochemical mutation on the birulence of bacterium typhosum: The loss of virulence of certain mutants. Br J Exp Path 1951; 32: 85-96. 39. Bacon G A, Burrows T W, Yates M. The effects of biochemical mutation on the virulence of bacterium typhosum: The virulence of mutants. Br J Exp Path 1950; 31: 714-724.
40. Bacon, G. A., Burrows, T. W., Yates, M. The effects of biochemical mutation on the virulence of bacterium typhosum: the induction and isolation of mutants. Br J Exp Path 1950; 31: 703-713.
41. Pawelek J, Low K B, Bermudes D. Tumor-targeted Salmonella as a novel anti-cancer vector. Cancer Res 1997; 57: 4537-4544.
42. Luo, X., Li, Z., Lin, S., Le, T., Ittensohn, M., Bermudes, D., Runyan, J. D., Shen, S., Chen, J., King, I. C. and Zheng, L-m. Antitumor effect of VNP20009, an attenuated Salmonella, in murine tumor models. Oncology Research 2001; 12: 501-508.
43. Low K B, Ittensohn M, Le T, Platt J, Sodi S, Amoss M, Ash O, Carmichael E, Chakraborty A, Fischer J, Lin S L, Luo X, Miller S I, Zheng L M, King I, Pawelek J, Bermudes D. Lipid A mutant Salmonella with suppressed virulence and TNFα induction retain tumor-targeting in vivo. Nature Biotechnology 1999; 17: 37-41.
44. Bermudes, D., Low, B., and Pawelek, J. Tumor-targeted Salmonella: Highly selective delivery vectors. In N. Habib (ed), Cancer Gene Therapy, Plenum Press, 1999; 415-432.
45. Clairmont, C., Lee, K. C., Pike, J., Ittensohn, M., Low, K. B., Pawelek, J., Bermudes, D., Brecher, S. M., Margitich, D., Turnier, J., Li, Z., Luo, X., King, I., and Zheng, L. M. Biodistribution and genetic stability of the novel antitumor agent VNP20009, a genetically modified strain of Salmonella typhimurium. J Infec. Diseases 2000; 181: 1996-2002.
46. Bermudes, D, Low, B, and Pawelek, J. Tumor-targeted Salmonella: Strain development and expression of the HSV TK effector gene. In: Gene Therapy: Methods and Protocols, W. Walther and U. Stein eds. Humana Press, Totowa, N.J. 2000; 35: 419-436.
47. Bermudes, D., Low, K. B., Pawelek, J., Sznol, M., Belcourt, M., Zheng, L-m, and King, I. Tumor-specific Salmonella-based cancer therapy. Stephen E. Harding (ed) Biotechnology and Genetic Engineering Rev 2001; 18: 219-233.
48. Li, Z., Lang, W., Runyan, J., Luo, X., Clairmont, C., Shen, S., Bermudes, D., Lin, S., Chen, J., Trailsmith, M., King, I., and Zheng, L. M. Tissue distribution and in vivo expression of cytosine deaminase by TAPET-CD, a genetically engineered strain of Salmonella typhimurium as an anti-tumor vector. Am Assoc Cancer Re. 2001; 42: 687.
49. Lin, S. L., Spinka, T. L., Le, T. X., Pianta, T. J., King, I., Belcourt, M. F., Li, Z. Tumor-directed delivery and amplification of tumor-necrosis factor-alpha by attenuated Salmonella typhimurium. Clin Cancer Res 1999; 5: 3822s.
50. Clairmont, C., Troy, L., Luo, X., Li, Z., Zheng, L-M., King, I. and Bermudes, D. Expression of Colicin E3 by tumor targeted Salmonella, enhances anti-tumor efficacy. Proc Amer AssocCancer Res Ann Meeting 2000; No. 41: 466.
51. Hensel, M. Salmonella Pathogenicity Island 2. Mol Microbiol 2000; 36: 1015-1023.
52. Hensel, M., Shea, J. E., Waterman, S. R., Mundy, R., Nikolaus, T., Banks, G., Vazquez-Torres, A., Gleeson, C., Fang, F. C., and Holden, D. W. Genes encoding putative effector proteins of the type III secretion system of Salmonella pathogenicity island 2 are required for bacterial virulence and proliferation in macrophages. Mol Microbiol 1998; 30: 163-174.
53. Shea, J E, Hensel, M, Gleeson, C, and Holden, D W. Identification of a virulence locus encoding a second type III secretion system in Salmonella typhimurium. Proc Nat Acad Sci USA 1996; 93: 2593-2597.
54. Ochman, H, Soncini, F C, Solomon, F, and Groisman, E A. Identification of a pathogenicity island required for Salmonella survival in host cells. Proc Nat Acad Sci USA 1996; 93: 7800-7804.
55. Cirillo, DM, Valdivia, R H, Monack, D M, and Falkow, S. Macrophage-dependent induction of the Salmonella pathogenicity island 2 type III secretion system and its role in intracellular survival. Mol Microbiol 1998; 30: 175-188.
56. Lostroh, C P, Bajaj, V, and Lee, C A. The cis requirements for transcriptional activation by HilA, a virulence determinant encoded on SPI 1. Mol Microbiol 2000; 37: 300-315.
57. Hensel, M. Functional analysis of ssaJ and the ssaK/U operon, 13 genes encoding components of the type III secretion apparatus of Salmonella pathogenicity island 2. Mol Microbiol 1997; 24: 155-167.
58. Deiwick, J., Nikolaus, T., Shea, J. E., Gleeson, C., Holden, D. W., and Hensel, M. Mutations in Salmonella pathogenicity island 2 (SPI2) genes affecting transcription of SPI1 genes and resistance to antimicrobial agents. J Bacteriol 1998; 180: 4775-4780.
59. Wilson, R. W., Ballantyne, C. M., Smith, C. W., Montgomery, C., Bradley, A., O'Brien, W. E., and Beaudet, A. L. Gene targeting yields a CD18⁻ mutant mouse for study of inflammation. J Immunol 1993; 151: 1571-1578.
60. Vazquez-Torres A., Jones-Carson, J., Baumlert, A. J., Falkow, S., Valdivia, R., Brown, W., Le, M., Berggren, R., Parks, W. T., and Fang, F. C. Extraintestinal dissemination of Salmonella by CD18-expressing phagocytes. Nature 1999; 401: 804-808.
61. Uchiya, K-i. Barbieri, M. A., Funato, K., Shah, A. H., Stahl, P. D., and Groisman, E. A. A Salmonella virulence protein that inhibits cellular trafficking. EMBO J. 1999; 18: 3924-3933.
62. Pawelek, J. M., Sodi, S., Chakraborty, A. K., Platt, J. T., Miller, S., Holden, D. W., Hensel, M., and Low, K. B. Salmonella Pathogenicity Island-2 and anticancer activity in mice. Cancer Gene Ther. 2002; 9: 813-818.
63. Bone, R. C. Toward an epidemiology and natural history of SIRS (systemic inflammatory response syndrome) JAMA 1992; 268:3452-3455.
64. Dinarello, C. A., Gelfand, J. A., and Wolff, S. M. Anticytokine strategies in the treatment of the systemic inflammatory response syndrome. JAMA 1993; 269:1829-1835.
65. Somerville, J. E., Cassaiano Jr., L., Bainbridge, B., Cunningham, M. D., and Darveau, R. P. A novel Escherichia coli lipid A mutant that produces an antiinflammatory lipopolysaccharide. J Clin Inves. 1996;. 97: 359-365.
66. Clementz, T., Zhou, Z., and Raetz, C. R. H. Function of the Escherichia coli msbB gene, a multicopy suppressor of htrB knockouts, in the acylation of lipid A. J Biol Chem 1997; 272: 10353-10360.
67. Khan, S. A., Everest, P., Servos, S., Foxwell, N., Zahringer, U., Brade, H., Rietschel, E. Th., Dougan, G., Charles, I. G., and Maskell, D. J. A lethal role for lipid A in Salmonella infections. Mol Mircobiol 1998; 29: 571-579.
68. Murray S R, Bermudes D, de Felipe Kans., Low K B. Extragenic suppressors of growth defects in msbB Salmonella. J Bacteriol 2001; 183: 5554-61.
69. Toso. J. F., Gill, V. J., Hwu, P., Marincola, F. M., Restifo, N. P., Schwartzentruber, D. J., Sherry. R. M., Topalian, S. L., Yang, J. C., Stock, F., Freezer, L J., Morton, K. E., Seipp, Cl, Haworth, L., Mavroukakis, S., White, D., MacDonald, S., Mao, J., Sznol, M., and Rosenberg, S. Phase I study of the intravenous administration of attenuated Salmonella typhimurium to patients with metastatic melanoma. J Clin Oncol 2002; 20: 142-152.
70. Rosenberg, S. A., Spiess, P. M., and Kleiner, D. E. Antitumor effects in mice of the intravenous injection of attenuated Salmonella typhimurium. J Immunothers 2002; 25: 218-225.
71. Garapin A. C., Colbère-Garapin, F, Cohen-Solal, Horondniceanu, F. and Kourilsky, P. Expression of herpes simples virus type I thymidine kinase gene in Escherichia coli. Proc Natl Acad Sci USA 1981; 78: 815-819
72. Tjuvajev, J., Blasberg, R., Luo, X., Zheng, L.-M., King, I., and Bermudes, D. Salmonella-Based Tumor-Targeted Cancer Therapy: Tumor Amplified Protein Expression Therapy (TAPET™) For Diagnostic Imaging. J Controlled Release 2001; 74: 313-315.
73. Platt, J., Sodi, S., Kelley, M., Rockwell, S., Bermudes, D., Low, K. B., and Pawelek, J. Antitumor effects of genetically engineered Salmonella in combination with radiation. Eur J Cancer. 2000; 36: 2397-2402.
74. Peters, L. J., Byers, R. M., Ang, K. K. Radiotherapy for Melanoma in Cutaneous Melanoma, Second Edition (eds Balch, C. M., Houghton, A. N., Milton, G. W., Sober, A. J., and Soong, S-j) J. B. Lippincott Co, Phila; 1992; pp 509-521.

Bordetella pertussis causes whooping cough (pertussis) B. pertussis is a very small Gram-negative aerobic coccobacillus that appears singly or in pairs. Its metabolism is respiratory and non-fermentative. Bordetella pertussis colonizes the cilia of the mammalian respiratory epithelium. Generally, B. pertussis has been considered non-invasive, although it can be sequestered in alveolar macrophages. The bacterium is a pathogen for humans and possibly for higher primates, and no other reservoir is known. Humans are regularly immunized against B. pertussis, to prevent whooping cough.

The disease pertussis has two stages. The first stage, colonization, is an upper respiratory disease with fever, malaise and coughing, which increases in intensity over about a 10-day period. During this stage the organism can be recovered in large numbers from pharyngeal cultures, and the severity and duration of the disease can be reduced by antimicrobial treatment. Adherence mechanisms of B. pertussis involve a “filamentous hemagglutinin” (FHA), which is a fimbrial-like structure on the bacterial surface, and cell-bound pertussis toxin (PTx). Short range effects of soluble toxins may also play a role as in invasion during the colonization stage. The second or toxemic stage of pertussis follows relatively nonspecific symptoms of the colonizaton stage. It begins gradually with prolonged and paroxysmal coughing that often ends in a characteristic inspiratory gasp (whoop). During the second stage, B. pertussis can rarely be recovered, and antimicrobial agents have no effect on the progress of the disease. This stage is mediated by a variety of soluble toxins. Although pertussis toxin is synthesized solely by B. pertussis, both B. parapertussis and B. bronchiseptica possess genes for pertussis toxin without expressing them. Bordetella parapertussis expresses pertussis toxin when the toxin gene from the B. pertussis chromosome is introduced into B. parapertussis.

Studies of B. pertussis and its adhesins have focused on cultured mammalian cells that lack most of the features of ciliated epithelial cells. However, some generalities have been drawn. The two most important colonization factors are the filamentous hemagglutinin (FHA) and the pertussis toxin (PTx). Filamentous hemagglutinin is a large (220 kDa) protein that forms filamentous structures on the cell surface. FHA binds to galactose residues on a sulfated glycolipid called sulfatide which is very common on the surface of ciliated cells. Mutations in the FHA structural gene reduce the ability of the organism to colonize, and antibodies against FHA provide protection against infection. However, other adhesions besides FHA may be involved in colonization. The structural gene for FHA has been cloned and expressed in E. coli, leading to its production for use in the acellular (component) vaccine.

One of the toxins of B. pertussis, the pertussis toxin (PTx), is also involved in adherence to the tracheal epithelium. Pertussis toxin is a 105 kDa protein composed of six subunits: SI, S2, S3, S4 (×2), and S5. The toxin is both secreted into the extracellular fluid and cell bound. Some components of the cell-bound toxin (S2 and S3) function as adhesins, and appear to bind the bacteria to host cells. S2 and S3 utilize different receptors on host cells. S2 binds specifically to a glycolipid called lactosylceramide, which is found primarily on the ciliated epithelial cells. S3 binds to a glycoprotein found mainly on phagocytic cells.

The S1 subunit of pertussis toxin is the A component with ADP ribosylating activity, and the function of S2 and S3 is presumed to be involved in binding the intact (extracellular) toxin to its target cell surface. Antibodies against PTx components prevent colonization of ciliated cells by the bacteria and provide effective protection against infection. Thus, pertussis toxin is clearly an important virulence factor in the initial colonization stage of the infection.

Since the S3 subunit of pertussis toxin is able to bind to the surface of phagocytes, and since FHA will attach to integrin CR3 on phagocyte surfaces (the receptor for complement C3b), it is possible that the bacterium might bind preferentially to phagocytes in order to facilitate its own engulfment. Bacteria taken up by this abnormal route may avoid stimulating the oxidative burst that normally accompanies phagocytic uptake of bacterial cells which are opsonized by antibodies or complement C3b. Once inside of cells the bacteria might utilize other toxins (i.e. adenylate cyclase toxin) to compromise the bactericidal activities of phagocytes. Bordetella pertussis may use this mechanism to get into and to persist in phagocytes as an intracellular parasite. B. pertussis produces at least two other types of adhesins, two types of fimbriae and a nonfimbrial surface protein called pertactin.

B. pertussis produces a variety of substances with toxic activity in the class of exotoxins and endotoxins. It secretes its own invasive adenylate cyclase (AC) which enters mammalian cells (Bacillus anthracis produces a similar enzyme, EF). This toxin acts locally to reduce phagocytic activity and probably helps the organism initiate infection. Pertussis A C is a 45 kDa protein that may be cell-associated or released into the environment. Mutants of B. pertussis in the adenylate cyclase gene have reduced virulence in mouse models. The organisms can still colonize but cannot produce the lethal disease. The adenylate cyclase toxin is a single polypeptide with an enzymatic domain (i.e., adenylate cyclase activity) and a binding domain that will attach to host cell surfaces. The adenylate cyclase was originally identified as a hemolysin. It may act by inserting into the erythrocyte membrane, causing hemolysis. The adenylate cyclase toxin is active only in the presence of a eukaryotic regulatory molecule called calmodulin, which up-regulates the activity of the eukaryotic adenylate cyclase. The adenylate cyclase toxin is only active in the eukaryotic cell since no similar regulatory molecule exists in prokaryotes, and appears to have evolved specifically to parasitize eukaryotic cells. Anthrax EF (edema factor) is also a calmodulin-dependent adenylate cyclase.

It produces a highly lethal toxin (formerly called dermonecrotic toxin) which causes inflammation and local necrosis adjacent to sites where B. pertussis is located. The lethal toxin is a 102 kDa protein composed of four subunits, two with a MW of 24 kDa and two with MW of 30 kDa. It causes necrotic skin lesions when low doses are injected subcutaneosly in mice and is lethal in high doses.

It also produces a substance called the tracheal cytotoxin, which is toxic for ciliated respiratory epithelium and which will stop the ciliated cells from beating. This substance is not a classic bacterial exotoxin since it is not composed of protein. The tracheal cytotoxin is a peptidoglycan fragment, which appears in the extracellular fluid where the bacteria are actively growing. The toxin kills ciliated cells and causes their extrusion from the mucosa. It also stimulates release of cytokine IL-1, and so causes fever.

It further produces the pertussis toxin, PTx, a protein that mediates both the colonization and toxemic stages of the disease. PTx is a two component, A+B bacterial exotoxin. The A subunit (S1) is an ADP ribosyl transferase. The B component, composed of five polypeptide subunits (S2 through S5), binds to specific carbohydrates on cell surfaces. PTx is transported from the site of growth of the Bordetella to various susceptible cells and tissues of the host. Following binding of the B component to host cells, the A subunit is inserted through the membrane and released into the cytoplasm in a mechanism of direct entry. The A subunit gains enzymatic activity and transfers the ADP ribosyl moiety of NAD to the membrane-bound regulatory protein Gi that normally inhibits the eukaryotic adenylate cyclase. The Gi protein is inactivated and cannot perform its normal function to inhibit adenylate cyclase. The conversion of ATP to cyclic AMP cannot be stopped and intracellular levels of cAMP increase. This has the effect to disrupt cellular function, and in the case of phagocytes, to decrease their phagocytic activities such as chemotaxis, engulfment, the oxidative burst, and bacteridcidal killing. Systemic effects of the toxin include lymphocytosis and alteration of hormonal activities that are regulated by cAMP, such as increased insulin production (resulting in hypoglycemia) and increased sensitivity to histamine (resulting in increased capillary permeability, hypotension and shock).

PTx also affects the immune system in experimental animals. B cells and T cells that leave the lymphatics show an inability to return. This alters both AMI and CMI responses and may explain the high freqency of secondary infections that accompany pertussis (the most frequent secondary infections during whooping cough are pneumomia and otitis media).

Although the effects of the pertussis toxin are dependent on ADP ribosylation and increases in cAMP, it has been shown that mere binding of the B oligomer of PTx can elicit a response on the cell surface such as lymphocyte mitogenicity, platelet activation, and production of insulin effects.

Adenylate cyclase (AC) is activated normally by a stimulatory regulatory protein (Gs) and guanosine triphosphate (GTP); however the activation is normally brief because an inhibitory regulatory protein (Gi) hydrolyzes the GTP. The cholera toxin A1 fragment catalyzes the attachment of ADP-Ribose (ADPR) to the regulatory protein Gs, forming Gs-ADPR from which GTP cannot be hydrolyzed. Since GTP hydrolysis is the event that inactivates adenylate cyclase (AC), the enzyme remains continually activated. The pertussis A subunit transfers the ADP ribosyl moiety of NAD to the membrane-bound regulatory protein Gi that normally inhibits the eukaryotic adenylate cyclase. The Gi protein is inactivated and cannot perform its normal function to inhibit adenylate cyclase. The conversion of ATP to cyclic AMP cannot be stopped.

As a Gram-negative bacterium, Bordetella pertussis possesses lipopolysaccharide (endotoxin) in its outer membrane, but its LPS is unusual. It is heterogeneous, with two major forms differing in the phosphate content of the lipid moiety. The alternative form of Lipid A is designated Lipid X. The unfractionated material elicits the usual effects of LPS (i.e., induction of IL-1, activation of complement, fever, hypotension, etc.), but the distribution of those activities is different in the two forms of LPS. For example, Lipid X, but not Lipid A, is pyrogenic, and its O-side chain is a very powerful immune adjuvant. Furthermore, Bordetella LPS is more potent in the limulus assay than LPS from other Gram-negative bacteria, so it is not reliable to apply knowledge of the biological activity of LPS in the Enterobacteriaceae to the LPS of Bordetella. The role of this unusual LPS in the pathogenesis of whooping cough has not been investigated. B. pertussis is regulated in different ways. Expression of virulence factors is regulated by the bvg operon. First, the organisms can undergo an event called phase variation resulting in the loss of most virulence factors and some undefined outer membrane proteins. Phase variation has been shown to occur at a genetic frequency of 10-4-10-6 generations and results from a specific DNA frame shift that comes about after the insertion of a single nucleotide into the bvg operon.

A similar process called phenotypic modulation, occurs in response to environmental signals such as temperature or chemical content, and is reversible. This is an adaptive process mediated by the products of the bvg operon, and is an example of a two-component environmental-sensing (regulatory) system used by many bacteria. The expression of these regulatory proteins is itself regulated by environmental signals, such that entry into a host might induce components required for survival and production of disease.

A number of references are cited within this specification, the entire disclosures of which are incorporated herein, in their entirety, by reference. See:
http://www.bact.wisc.edu/Bact330/lecturebpertussis
http://gsbs.utmb.edu/microbook/ch031.htm
http://www.sanger.ac.uk/Projects/B_pertussis/(e.g., B. pertussis genetic sequence)
http://www.sanger.ac.uk/Projects/B_parapertussis/(e.g., B. parapertussis genetic sequence)
http://www.sanger.ac.uk/Projects/B_bronchiseptica/(e.g., B. bronchiseptica genetic sequence) JP63-44529 (Feb. 25, 1988)
Alonso S, Pethe K, Mielcarek N, Raze D, Locht c. Role of ADP-ribosyltransferase activity of pertussis toxin in toxin-adhesin redundancy with filamentous gemagglutinin during Bordetella pertussis infection. Infection & Immunity 69(10):6038-6043, 2001.
Alouf J E, Freer J H: Sourcebook of bacterial protein toxins. Academic Press, London, 1991
Arai H. Munoz J: Crystallization of pertussigen from Bordetella pertussis. Infect Immun 311:495, 1981
Bachelet M, Richard M, Francois D, Polla B S. Mitochondrial alterations precede Bordetella pertussis-induced apoptosis. FEMS Immunology and Medical Microbiology 32:125-131, 2002.
Barbic J, Leef M, Burns D L, Shahin R D. Role of Gamma Interferon in Natural Clearance of Bordtella pertussis Infection. Infection and Immunity 65(12):4904-4908, 1997.
Bassinet L, Gueirard P, Maitre B, Housset B, Gounon P, Guiso N. Role of Adhesins and Toxins in Invasion of Human Tracheal Epithelial Cells by Bordetella pertussis. Infection and Immunity 68(4):1934-1941, 2000.
Bemis D A, Kennedy J R: An improved system for studying of Bordetella bronchiseptica on the ciliary activity of canine tracheal epithelial cells. J Infect Dis 144:349, 1981
Blondiau C, Lagadec P, Lejeune P, Onier N, Cavaillon J M, Jeannin J F. Correlation between the capacity to activate macrophages in vitro and the antitumor activity in vivo of lipopolysaccharides from different bacterial species. Immunobiology 190(3):243-254, 1994.
Bemis D A, Kennedy J R: An improved system for studying of Bordetella bronchiseptica on the ciliary activity of canine tracheal epithelial cells. J Infect Dis 144:349, 1981
Bordet J. Gengou U: Le microbe de la coqueluche. Ann Inst Pasteur 20:731, 1906 Casano P. Pons Odena M, Cambra F J, Martin J M, Palomeque A. Bordetella pertussis infection causing pulmonary hypertension. Archives of Disease in Childhood 86(6):453, 2002.
Chouakri O, Ktari F, Lavaud J, Maury I, Lode N, Durand S, Chabernaud J L, Arbaoui H, Lemouchi A, Barbier M L. [Severe bacterial infections in children. Survey by the pediatric mobile intensive care unit AP/HP in the Ile-de-France area]. Arch Pediatr 8(4):712s-720s, 2001.
Clark V L, Bavoil P M. Bacterial Pathogenesis. Interaction of Pathogenic Bacteria with Host Cells. Methods in Enzymology 236:332-345, 1994.
Codeco C T, Luz P M. Is pertussis actually reemerging? Insights from an individual-based model. Cad Saud Publica 17(3):491-5000, 2001.
Colquhoun J P. Children's Corner. Australian Family Physician 26(7):875, 1997.
Coote J G. Environmental Sensing Mechanisms in Bordetella. Advances In Microbial Physiology 44:141-181, 2001.
Doyle R J. Adhesion of Microbial Pathogens. Methods in Enzymology 253:3-13, 1995.
Duclos P, Olive J. Invited Commentary: Pertussis, A Forgotten Killer. American Journal of Epidemiology 155(10):897-898, 2002.
Ewanowich C A, Melton A R, Weiss A A, Sherburne R K, Peppler Miss. Invasion of HeLa 229 Cells by Virulent Bordetella pertussis. Infection And Immunity 57(9):2698-2704, 1989.
Ewanowich C A, Shervurne R K, Man S FP, Peppler Miss. Bordetella parapertussis Invasion of HeLa 229 Cells and Human Resopiratory Epithelial Cells in Primary Culture. Infection And Immunity 75(4):1240-1247, 1989. Finger H, Heymer B, Hof H et al: Ueber Struktur und biologische Aktivitat von Bordetella pertussis Endotoxin. Zentralbl Bakteriol Mikrobiol Hyg 1 Abt OrigA 235:56, 1976
Finger H, Wirsing von Koenig C H: Enhancement and suppression of immune responsiveness by bacteria, bacterial products and extracts. p. 16. In Zschiesche W (ed): Immune Modulation by Infectious Agents. Gustav Fischer Verlag, Jena, 1987
Finger H, Wirsing von Koenig C H: Serological diagnosis of whooping cough. Dev Biol Stand 61:331, 1985
Friedman R L, Nordensson K, Wilson L, Akporiaye E T, Yocum D E. Uptake and Intracellular Survival of Bordetella pertussis in Human Macophages. Infection and Immunity 60(11):4578-4585, 1992.
Goldman W E, Klapper D G, Basemann J B: Detection, isolation and analysis of a released Bordetella pertussis product toxic to cultured tracheal cells. Infect Immun 36:782, 1982
Goodman Y E, Wort A J, Jackson F L: Enzyme-linked immunosorbent assay for detection of pertussis immunoglobulin A in nasopharyngeal secretions as an indicator of recent infection. J Clin Microbiol 13:286, 1981
Guermonprez P, Khelef N, Blouin E, Rieu P, Ricciardi-Castagnoli P, Guiso N, Ladant D, Leclerc C. The Adenylate Cyclase Toxin of Bordetella pertussis Binds to Target Cells via the MB2 Integrin (CD11 b/CD18). The Journal of Experimental Medicine 193(9):1035-1044, 2001.
Hazenbos W L W, van den Berg B M, vna't Wout J W, Mooi F R, van Furth R. Virulence Factors Determine Attachment and Ingestion of Nonopsonized an dOpsonized Bordetella pertussis by Human Monocytes. Infection and Immunity 62(11):4818-4824, 1994.
Heiniger U. Pertussis: an old disease that is still with us. Curr Opin Infect Dis 14(3):329-335, 2001.
Hitchcock P J, Leive L, Makela P H, Rietschel E T, Strittmatter W, Morrison D C. Journal Of Bacteriology 166(3):699-705, 1986.
Hof H, Emmerling P, Hacker J, Hughes C. The Role Of Macrophages In Primary And Secondary Infection Of Mice With Salmonella Typhimurium. Ann. Immunol. 133 c:21-32, 1982.
Horowitz Y, Greenberg D, Ling G. Lifshitz M. Acrodynia: a case report of two siblings. Arch Dis Child 86:453-455, 2002.
Janda W M, Santos E, Stevens J, Celig D, Terrile L, Schreckenberger P C. Unexpected isolation of Bordetella pertussis from a blood culture. J Clin Microbiol 32(11):2851-8253, 1994.
Keer J R, Preston N W. Current pharmacotherapy of pertussis. Expert Opin Pharmacother 2(8):1275-1282, 2001.
Kersters K, Hinz K-H, Hertle A et al: Bordetella avium sp. nov., isolated from the respiratory tract of turkeys and other birds. Int J Syst Bacteriol 34:56, 1984
Khelef N. Gounon P, Guiso N. Internalization of Bordetella pertussis adenylate cyclase-haemolysin into endocytic vesicles contributes to macrophage cytotoxicity. Cellular Microbiology 3(111):721-730, 2001.
Konda T, Kamachi K, Iwaki M, Matsunaga Y. Distribution of pertussis antibodies among different age groups in Japan. Vaccine 20:1711-1717, 2002.
Le Dur A, Chaby R, Szabo L. Isolation of Two Protein-Free and Chemically Different Lipopolysaccharides from Bordetella pertussis Phenol-Extracted Endotoxin. Journal Of Bacteriology 143(1):78-88, 1980.
Lee C K, Roberts A L, Finn T M, Knapp S, Mekalanos J J. A New Assay for Invasion of Hela 229 Cells by Bordetella pertussis: Effects of Inbibitors, Phenotypic Modulation, and Gneetic Alterations. Infection And Immunity 58(8):2516-2522, 1990.
Leef M, Elkins K L, Barbic J. Shahin R D. Protective Immunity to Bordetella pertussis Requires Both B Cells and CD4+ T Cells for Key Functions Other than Specific Anibody Production. The Journal of Experimental Medicine 191(11):1841-1852, 2000.
MacLean D W. Bordetella Pertussis Infection In Patients With Bronchogenic Carcinoma. The Lancet Feb. 28, 1981.
Mahon B P, Sheahan B J, Griffin F, Murphy G, Mills K HG. A typical Disease after Bordetella pertussis Respiratory Infection of Mice with Targeted Disruptions of Interferon-Y Receptor or Immunoglobulin m Chain Genes. J. Exp. Med. 186(11):1843-1851.
Masure H R. The adenylate cyclase toxin contributes to the survival of Bordetella pertussis within human macrophages. Microbial Pathogenesis 14:253-260, 1993.
McGuirk P, Mahon B P, Griffin F, Mills K HG. Article: Compartmentalization of T cell responses following respiratory infection with Bordetella pertussis: hyporesponsiveness of lung T cells is associated with modulated expression of th eco-stimulatory molecule CD28. European Journal of Immunology 28:153-163.
Meade B D, Mink C M, Manclark C R: Serodiagnosis of pertussis, p. 322. In: Manclark C R: Proc. 6th Intl Symp Pertussis, DHHS (FDA) Publication No. 90-1164; Bethesda, Md., 1990
Meade B D. Bollen A: Recommendations for use of the polymerase chain reaction in the diagnosis of Bordetella pertussis infections. J Med Microbiol 41:51-55, 1994
Mills K H G. Immunity to Bordetella pertussis. Microbes and Infection 3:655-677, 2001.
Mills K H G. Barnard A, Watkins J. Redhead K. Cell-Mediated Immunity to Bordetella pertussis: Role of Th1 Cells in Bacterial Clearance in a Murine Respiratory Infection Model. Infection and Immunity 61(2):399-410, 1993.
Monack D, Munoz J J, Peacock M G et al: Expression of pertussis toxin correlates with pathogenesis in Bordetella species. J Infect Dis 159:205, 1989
Mooi F R, Loo I H, King A J. Adaptation of Bordetella pertussis to vaccination: a cause for its reemergence? Emerg Infect Dis 7(3):526-528, 2001.
Mouallem M, Farfel Z, Hanski E. Bordetella pertussis Adenylate Cyclase Toxin: Intoxication of Host Cells by Bacterial Invasion. Infection and Immunity 58(11):3759-3764, 1990.
Munoz J J, Bergman R K: Bordetella pertussis. Vol. 4. Marcel Dekker, New York, 1977
Ohnishi M, Kimura S, Yamazaki M, Oshima H, Mizuno D I, Abe S, Yamaguchi H. Anti-tumor activity of low-toxicity lipopolysaccharide of Bordetella pertussis. Br J Cancer 69(6):1038-1042, 1994.
Oldenburg D J, Storm D R. Identification of a domain in Bordetella pertussis adenylyl cyclase important for subunit interactions and cell invasion activity. Microb Path 15(2):153-157, 1993.
Oldenburg D J, Gross M K, Wong C S, Storm D R. High-Affinity Calmodulin Binding Is Required for the Rapid Entry of Bordetella pertussis Adenylyl Cyclase into Neuroblastoma Cells. Biochemistry 31:8884-8891, 1992.
Pawelek, J. M., Low, K. B., Bermudes, D., Bacteria As Tumor-Targeting Vectors, Lancet Oncology Review (MS. 02ONCL/607) in press, 2003
Pittman M: Pertussis toxin: the cause of the harmful effects and prolonged immunity in whooping cough. A hypothesis. Rev Infect Dis 1:402, 1979
Pittman M: The concept of pertussis as a toxin-mediated disease. Pediatr Infect Dis J 3:467. 1984
Poynten M, Hanlon M, Irwig L, Gilbert G L. Serological diagnosis of pertussis: evaluation of IgA against whole cell and specific Bordetella pertussis antigens as markers of recent infection. Epidemiol Infection 128(2):161-167, 2002.
Prasad S M, Yin Y, Rodzinski E, Tuomanen E I, Masure H R. Identificaiton of a carbohydrate recognition domain in filamentous hemnagglutinin from Bordetella pertussis. Infect Immun 61(7):2780-2785, 1993.
Preziosi M, Yam A, Wassilak S GF, Chabirand L, Simaga A, Ndiaye M, Dia M, Dabis F, Simondon F. Epidemiology of Pertussis in a West African Community before and after Introduction of a Widespread Vaccination Program. American Journal of Epidemiology 155(10):891-896, 2002.
Purnell D M, Bartlett G L, Kreider J W, Biro T G, Knotra J. Comparative Antitumor Effects of Corynebacterium parvum, Bordetella pertussis, Bacillus Calmette-Guerin, and Levamisole Alone or in Combination with Cyclophosphamide in the CaD2 Murine Mmamary Adenocarcinoma System. Cancer Research 39:4838-4842, 1979.
Purnell D M, Kreider J W, Bartlett G L. Evaluation of Antitumor Activity of Bordetella pertussis in Two Murine Tumor Models. Journal Of The National Cancer Institute 55(1):123-128, 1975.
Redhead K, Watkins J, Barnard A, Mills K HG. Effective Immunization against Bordetella pertussis Respiratory Infection in Mice Is Dependent on Induction of Cell-Mediatd Immunity. Infection And Immunity 61(8):3190-3198, 1993.
Relman D, Tuomanen E, Falkow S, Golenbock D T, Saukonen K, Wright S.Dak. Recognition of a Bacterial Adhesin by an Integrin: Macrophage CR3 (amB2, CD11b/CD18) Binds Filamentous Hemagglutinin of Bordetella pertussis. Cell 61:1375-1382, 1990.
Robinson A, Duggleby C J, Gorringe A R et al: Antigenic variation in Bordetella pertussis. p. 147. In Birbeck T H, Penn C W (eds): Antigenic Variation and Infectious Diseases. Society for General Microbiology, IRL Press, Oxford, 1986
Rozdzinski E, Tuomanen E. Adhesion of Microbial Pathogens to Leukocyte Integrins: Methods of Study Ligand Mimicry. Methods in Enxymology 253:3-13, 1995.
Sanz Moreno J C Ed Ory Manchon F. Gonzalez Alonso J, La Torre J L, Salmeron F, Limia A, Tello O, Pachon I, Amela C, Vazquez J, Ory Fd F, Sanz J C. Laboratory diagnosis of Pertussis. Role of the serology. Enferm Infecc Microbiol Clin 20(5):212-218, 2002.
Sato Y, Sato H. Animal Models of Pertussis. Pathogenesis and Immunity in Pertussis Chapter 15:309-325, 1988.
Saukkonen K, Burnette W N, Mar V L, Masure H R, Tuomanen E I. Pertussis toxin has eukaryotic-like carbohydrate recognition domains. Proc. Natl. Acad. Sci. USA 89:118-122, 1992.
Saukkonen K, Cabellos C, Burroughs M, Prasad S, Tuomenen E. Integrin-mediated Localization of Bordetella pertussis within Macrophages: Role in Pulmonary Colonization. J. Esp. Med. 173:1143-1149, 1991.
Schaeffer L M, Weiss A A. Pertussis Toxin and Lipopolysaccharide Influence Phagocytosis of Bordetella pertussis by Human Monocytes. Infection And Immunity 69(12):7635-7641, 2001.
Schmidt-Schlapfer G, Liese J G, Porter F, Stojanov S, Just M, Belohradsky B H. Polymerase chain reaction (PCR) compared with conventional identificaiton in culture for detection of Bordetella pertussis in 7153 children. Clin Microbiol Infect 3(4):462-467, 1997.
Shevliagin VIa, Borodina N P, Snegireva A E, Shaposhnikova G M. Infectious risk factors in the development of malignant neoplasms. Zh Mikrobiol Epidermiol Immunobiol 4:40-42, 1995.
Steed L L, Akporiaye E T, Friedman R L. Bordetella pertussis Induces Respiratory Burst Activity in Human Polymorphonuclear Leukocytes. Infection and Immunity 60(5):2101-2105. 1992.
Steed L L, Setareh, Friedman R L. Intracellular Survival of Virulent Bordetella pertussis in Human Polymorphonuclear Leukocytes. Journal fo Leukocyte Biology 50:321-330, 1991.
Tahri-Jouti M, Chaby R. Specific Binding Of Lipopolysaccharides to Mouse Macrophages-I. Characteristics Of The Interaction And Ineffeciency Of the Polysaccharide Region. Molecular Immunology 27(8):751-761, 1990.
Tuomanen E. Subversion of Leukocyte Adhesion Systems by Respiratory Pathogens. ASM News 59(6):292-296, 1992.
Tuomanen E I, Nedelman J, Hendley J O, Hewlett E L. Species Specificity of Bordetella Adherence to Human and Animal Ciliated Respiratory Epithelial Cells. Infection and Immunity 42(2):692-695, 1983.
Urisu A, Cowell J L, Manclark C R. Involvement of filamentous hemagglutinin in the adherence of Bordetella pertussis to human WiDr cell cultures. Developments in Biological Standardization 61:205-214, 1985.
Vandamme P. Hommez J. Vancanneyt M, Monsieurs M, Hoste B, Cookson B T, Wirsing von Konig C H, Kersters K, Blackall P J: Bordetella hinzii sp. nov. isolated from poultry and humans. Int J Syst Bact 45:37-45, 1995
Versteegh F G, Schellekens J F, Nagelkerke A F, Roord J J. Laboratory-confirmed reinfections with Bordetella pertussis. Acta Paediatr 91(1):95-97, 2002.
Wardlaw A C, Parton R: Pathogenesis and Immunity in Pertussis. John Wiley & Sons, New York. 1988
Watanabe M, Komatsu E, Abe K, lyama S, Sato T, Nagai M. Efficacy of pertussis components in an acellular vaccine, as assessed in a murine model of respiratory infection and a murine intracerebral challenge model. Vaccine 20:1429-1434, 2002.
Weingart C L, Weiss A A. Bordetella pertussis Virulence Factors Affect Phagocytosis by Human Neutrophils. Infection and Immunity 68(3):1735-1739, 2000. Weiss A A, Falkow S: Genetic analysis of phase change in Bordetella pertussis. Infect Immun 43:263, 1984
Wirsing von Koenig C H, Tacken A, Finger H: Use of supplemented Stainer-Scholte broth for the isolation of Bordetella pertussis from clinical material. J Clin Microbiol 26:2558, 1988
Wong W S, Simon D I, Rosoff P M, Rao N K, Chapman H A. Mechanisms of pertussis toxin-induced myelomonocytic cell adhesion: role of Mac-1 (CD11b/CD18) and urokinase receptor (CD87). Immunology 88(1):90-97, 1996.

### SUMMARY OF THE INVENTION

The present invention provides compositions and methods for targeting metastatic cells based on appearance of β1,6-branched oligosaccharides at the cell surface. The invention also provides for identification and targeting of cells believed to play a significant role in the progression of neoplasms, by their rather unique characteristics, which include β1,6-branched oligosaccharides and coarse vesicles, as well as other traits which will become more apparent herein. Human cancer cells have a widely expressed phenotype which includes expression of coarse vesicles rich in β1,6-branched oligosaccharides. β1,6-branching, catalyzed by GNT-V, is associated with metastasis, and predicts poor survival in primary human breast and colon carcinomas. In studies of β1,6-branching (determined by LPHA lectin-histochemistry) in 119 archival specimens of human melanomas and other neoplasms, including carcinomas of the lung, colon, breast, ovary, prostate, and kidney, most tumors (96%) stained to some extent with LPHA. Staining was always, but not exclusively, associated with coarse vesicles. In melanomas, LPHA staining co-localized with CD63, and gp100. In pigmented melanomas, the vesicles were melanized and are known as ‘coarse melanin’. LPHA-positive, i.e., β1,6-branched oligosaccharide containing, coarse melanin was a feature of both tumor cells and melanophages, and accounted for the well-known hypermelanotic regions of primary melanomas. LPHA-positive tumor cells varied widely in primaries (melanoma and others), ranging from 0-100% for a given tumor, while metastases were far more homogeneous (p=0.0080), with vesicular, LPHA-positive tumor cells comprising more than 75% of 15/16 metastatic melanomas and renal cell carcinomas. In studies by others, GNT-V elicited formation of autophagy-dependent, LPHA-positive vesicles in mink lung alveolar cells (1) (Hariri et al., Mol. Biol. Cell 11:255-268, 2000), suggesting that the coarse vesicles in tumors reported here may have been induced by GNT-V. Expression of the phenotype was so common and pervasive that it appeared to be an integral component of the biology of tumor progression. β1,6-branched oligosaccharides are normally expressed by myeoloid cells such as macrophages and granulocytes, are a prominent feature of experimental macrophage-melanoma hybrids (11). See, Tamara Handerson and John M. Pawelek, β1,6-branched oligosaccharides and coarse vesicles: A common, pervasive phenotype in melanoma and other human cancers, Cancer Research, in press, 2003, expressly incorporated herein by reference.

One hypothesis which explains the etiology of the 1,6-branched oligosaccharides is a hybrid hematopoetic origin of the cells which migrate distant from the site of the original tumor. Thus, if the primary tumor cells were fused with macrophages, this would explain a number of observations regarding metastatic cells, and also provide new insights into their diagnosis and treatments. See, Chakraborty, A., Lazova, R., Davies, S., Bäckvall, H., Ponten, F., Brash, D. and Pawelek, J., Genetic Evidence for Tumor-Hematopoietic Cell Hybrids in a Human Metastasis, submitted for publication (2003). In particular, since, according to this hypothesis, the cells express significant traits of macrophages, the biology of such cells may be used to influence their behavior. This exposes the opportunity to use specific growth factors, receptors, cell surface structures, and possibly bacterial and viral targeting mechanisms, to specifically treat pathology associated with these cells, with a fuller understanding of the biology of the cells, and the expected characteristics of the hybrids.

The ability to target these cells may be advantageously applied for the diagnosis, or treatment of disease, or determination of disease prognosis.

The targeting agent may be, for example, a pharmaceutical (e.g., small molecule), macromolecule, virus or organism. The targeting agent may be directly responsible for the desired result, or a part of a cascade, e.g., an initiator of a process. Other elements of the cascade may be endogenous to the organism or administered exogenously.

Therefore, it is an aspect of the invention to employ such aberrant oligosaccharides, and their corresponding protein, lipid, and glycosaminoglycan glycogonjugates, as molecular targets for metastatic disease and/or diagnostic imaging therefor.

β1,6-branched oligosaccharides on metastatic cancer cells represent generalizable molecular targets for treatment of metastatic disease. Thus, according to the present invention, oligosaccharide-targeting agents or vectors may be applied for treatment or diagnosis, for example by diagnostic imaging, of metastatic disease. These agents include, but are not limited to, bacteria, viruses, lectins, antibodies, and liposomes, each of which may exhibit specific binding capabilities for aberrant oligosaccharides, and/or their corresponding aberrant glycoconjugated proteins, lipids, and glycosaminoglycans on metastatic tumors. For treatment, it is preferred that the agent of vector bears inherent or engineered anticancer toxins, chemicals, or bioactive agents, and the like, to destroy cancer cells or otherwise inhibit tumor growth. For diagnosis, such attributes may be absent, and indeed may preferably be absent. On the other hand, a diagnostic agent, particularly for diagnostic imaging, comprises an attribute which itself, or in conjunction with another agent, provides a precise and accurate indication of a presence of the targeted attribute, e.g., oligosaccharide.

Likewise, metastatic tumor cells which are macrophage hybrids may also be targeted based on the fact that they express traits of both the solid tumor cell (primary tumor) and the macrophage, suggesting that therapies which have synergistic effects when dual-targeted may be employed. For example, if a cell expresses surface markers specific for both parent cell lines, then each marker may be recognized with an antibody or receptor-specific ligand. For example, fluorescent resonance energy transfer (FRET) detection methods may be used to diagnose the existence of such cells. In some cases, the FRET itself may be used as a therapy, especially where the metastasis is accessible to external illumination. Otherwise, the conjunction of both these antibodies or ligands on the same cell may then be used to target the cell for a specific therapy, for example by providing agents which are synergistically toxic when both are endocytosed, or which allow a particular reaction.

It should also be clear that a variety of cell surface markers, both intrinsically specific, and those whose combination is specific, may be employed to identify and target these cells. Such markers may include β1,6-branched oligosaccharides, or be distinct therefrom.

Therefore, it should be apparent that hybrid macrophage-tumor cells may be distinguished from normal tissues, and specifically targeted based on their rather unique phenotype, for example, surface β1,6-branched oligosaccharides. Likewise, as a result of the realization that the malignant cells are macrophage-derived, other targeting strategies may be employed as well, for example use of, or modulation of, hematopoetic growth factors.

In some instances, the diagnostic agent may have insufficient selectivity, and thus produce false positive readings as an indicator of the intended pathology. Therefore, such non-selective diagnostic agents may be used together with other agents, which together have a useful sensitivity and selectivity. Advantageously, the diagnostic agent and co-agent are both detected using the same technique, either together or at different times. For example, diagnostic images based on two different agents, which each have suboptimal selectivity, may be compared for correspondence, e.g., MRI, PET, CAT, gamma emission, etc. The technique may also rely on a joint interactive effect of two agents, with a single measurement, for example an enzyme-substrate interaction, fluorescent resonance energy transfer (FRET), etc.

It is noted that the β1,6-branched oligosaccharides are themselves relatively specific for the metastatic cells, and therefore these alone may serve as useful diagnostic and therapeutic targets.

It is particularly preferred, according to the present invention, to use live Bordetella pertussis as a targeting agent, e.g., for cells expressing β1,6-branched oligosaccharides at their surface, since this organism displays both high specificity for the metastatic cells, as well as lethality thererto.

It is also an aspect of the present invention that certain Bordetellae and non-Bordetellae bacterial species and subspecies can be genetically engineered to contain certain genes whose transcription products or bi-products can be utilized for detecting said bacteria within tumors. For example, the bacteria may be genetically modified by the insertion of the gene(s) for myoglobin, for example on a plasmid or integrated into the bacterial genome. The myoglobin phenotype may be normally expressed, or linked to a promoter gene associated with acquisition of the target of the bacteria. Myoglobin, and in particular its association with oxygen, can be detected through non-invasive techniques of magnetic resonance imaging (MRI) and magnetic resonance spectroscopy (MRS). Such genetic engineering techniques, and the various reporters, and active and/or toxic gene products which may be employed, as well known in the art.

According to the present invention, a bacteria that expresses the reporter may have inherent specificities for aberrant oligosaccharides and corresponding glycoconjugated proteins on cancer cells, and thus be useful as a diagnostic agent. On the other hand, this reporter technique may be used with outer bacteria, having other specificities or intended targets. Such detection would be useful during therapy to determine when, and to what extent, tumors become colonized by the therapeutic bacteria following their injection into a patient. Likewise, to the extent that such gene expression may be toxic, for example by enhancing free radical reactions in proximity to the tumor cells, these may be useful therapies as well.

By providing compositions which are pharmaceutically acceptable, while localizable, such as by magnetic resonance imaging, gamma scintillation, positron emission, specific fluorescence, or the like, a diagnostic tool is provided which can be administered to mammals for the purpose of detecting and locating metastatic cell clusters.

By providing compositions which are specifically targeted toward cell surface markers, and cytotoxic or otherwise capable of generating a reaction resulting in cell death or significant metabolic change, such compositions also form a rational basis for therapy of metastatic disease. For example, treatments targeting cells expressing β1,6-branched oligosaccharides may be efficacious in the treatment of tumor cells are derived from a cell type selected from the group consisting of a metastatic carcinoma, metastatic melanoma, brain tumor, lymphoma, and myelogenous leukemia.

According to a preferred embodiment of the invention, Bordetella pertussis is administered as a cytotoxic agent which specifically targets cells expressing β1,6-branched oligosaccharides.

It is also an aspect of the invention to provide a chemotherapeutic regimen in combination with the administration of the agent. For example, after administering live Bordetella pertussis, Bordetella parapertussis, or Bordetella bronchiseptica, after the Bordetella has triggered an appropriate response in the host, for example causing an inflammatory response which results in necrosis of the metastatic tissue, an antibiotic such as erythromycin, clarithromycin, and/or azithromycin may be used as a therapy. The antibiotic therefore acts as a “rescue” from less specific and possibly deleterious effects of the bacteria, and may be used on an as-needed or prophylactic basis. Indeed, the bacteria may be engineered to have a specific susceptibility to a particular antibiotic, thus allowing use of a narrow spectrum drug with lower incidence of side effects.

Agents may be administered to enhance the toxicity of the bacteria. For example, it has been found that histidine administration enhances the toxicity of B. pertussis in vivo, while lack of histidine markedly slows growth.

In cases where live bacteria are used in a diagnostic manner, wherein the specificity of the bacteria for the tissues is the crux of the diagnostic test, rather than invasion and/or colonization of the target tissues, the use of concurrent antibiotics may be indicated. That is, the predicted occurrence of adverse reactions and symptomatic infections may be reduced by administering an agent which prevents bacterial proliferation in close temporal proximity with the administration of the bacteria. The agent bacteria may be genetically engineered to include a particular susceptibility to a particular antibiotic, for example a narrow spectrum antibiotic. The bacteria may also be selected for susceptibility to an agent.

Likewise, in employing the agent in a diagnostic system, the agent may be secondarily tagged, with the tag being the basis for the diagnosis. Therefore, it is not necessary for the agent itself to be distinguishable, for example using a medical imaging technology.

According to a further aspect of the invention, magnetic resonance spectroscopy (MRS) is employed, analyzing the spectra of a primary breast tumor, to predict prognosis or metastatic potential. MRS may be responsive to glycosylation, e.g., presence of β1,6-branched N-glycans, of the tissues, allowing distinctions to be made in this regard. MRS is well known on the art, and need not be further discussed herein.

It is further aspect of the present invention that certain viruses, for example, adenoviruses, engineered to express similar oligosaccharide-attachment specificities are also useful as said anticancer vectors. In this case, the adenovirus includes a cytotoxic payload. Likewise, certain viruses, for example, adenoviruses, engineered to express similar oligosaccharide-attachment specificities, may be used in accordance with the present invention for diagnostic imaging of tissues expressing the respective oligosaccharides, e.g., tumors.

Likewise, a virus having a high target specificity for a specific cell type, as known in the art, may be created which induces the infected cell to express β1,6-branched oligosaccharides on their surface, and thereby be targeted by agents according to another aspect of the present invention. It may also be possible to identify or engineer a virus which is specific for neoplasms expressing β1,6-branched oligosaccharides or other macrophage associated marker, but having low affinity for macrophages themselves.

It is further a part of this invention that certain lectins, liposomes, antibodies, and the like, modified to express similar oligosaccharide-attachment specificities are also useful as said anticancer agents.

It is further a part of this invention that certain non-living agents, including but not restricted to antibodies, lectins, and liposomes, modified with similar oligosaccharide-attachment specificities are also useful for diagnostic imaging of tissues expressing respectively similar oligosaccharides, e.g., tumors.

According to another aspect of the invention, an in vitro (living) biopsy sample of the primary tumor, or the tumor itself in vivo, is subjected to an agent which is specific or has high affinity for β1,6-branched N-glycans. The tumor is then analyzed, for example using a light microscope or MRS, to determine affinity of the agent for the cells. Cells expressing higher levels of affinity are generally correlated with poorer prognosis. This method may also be used to predict response to therapy. If a tumor has a high affinity for a diagnostic agent which is specific for the β1,6-branched N-glycans, it is likely that a therapy specific for these cell surface markers. Low affinity in a diagnostic test may indicate a low response to the corresponding therapeutic agent. It is noted, however, that different tumors from the same source may respond differently.

### BRIEF DESCRIPTION OF THE DRAWINGS

FIG. 1 shows a graph representing 2-observer blinded scoring of tissue microarrays; and

FIG. 2 shows a graph of human lung carcinoma A549 tumor volume over time for control and B. pertussis treated mice.

### DETAILED DESCRIPTION OF THE INVENTION

### EXAMPLE 1

Experimental Results: Breast Carcinoma

Breast tissue microarrays of tumor primaries and metastases were stained with the lectin LPHA (leucocytic phytohemagglutinin), which delineates an aberrant form of glycosylation, known as beta 1,6-branched N-glycans. This type of glycosylation was previously associated with poor survival when detected in breast primaries, although nothing was known of the staining status of metastases. In contrast, the primaries (processed side-by-side) stained at a significantly lower intensity. This therefore provides a basis for distinguishing between primary and metatstatic tumors, and presumably for distinguishing between primary tumors with high metastatic potential and those which are more benign.

Human breast carcinomas thus appear to be candidates for B. pertussis therapy, or for other such oligosaccharide-targetting therapies. β1,6-branching by LPHA lectin-histochemistry in archival human specimens of 60 primary and metastatic melanomas, 59 diverse neoplasms, including carcinomas of the lung, colon, prostate, kidney and liver, and nearly 600 breast carcinomas on tissue microarrays, comprised of ‘node-positive’ primary tumors and matched tumor-positive lymph nodes were studied. The metastases in general stained with greater intensity than did the primary tumors. About 300 metastases and 500 node-positive primaries were scored, and a marked increase in staining of metastases was found, with >95% of the metastatic cells staining homogeneously, and at very high intensity. Staining was always, but not exclusively, associated with coarse vesicles. Breast carcinomas metastatic to the lymph node stained with LPHA at significantly greater intensity than did the primary tumors (p<0.0001). It is possible that the coarse vesicles in tumors reported here were induced by GNT-V. LPHA-positivity in breast carcinomas, melanomas and a variety of other human cancers revealed co-expression of cytoplasmic coarse vesicles and β1,6-branched N-glycans.

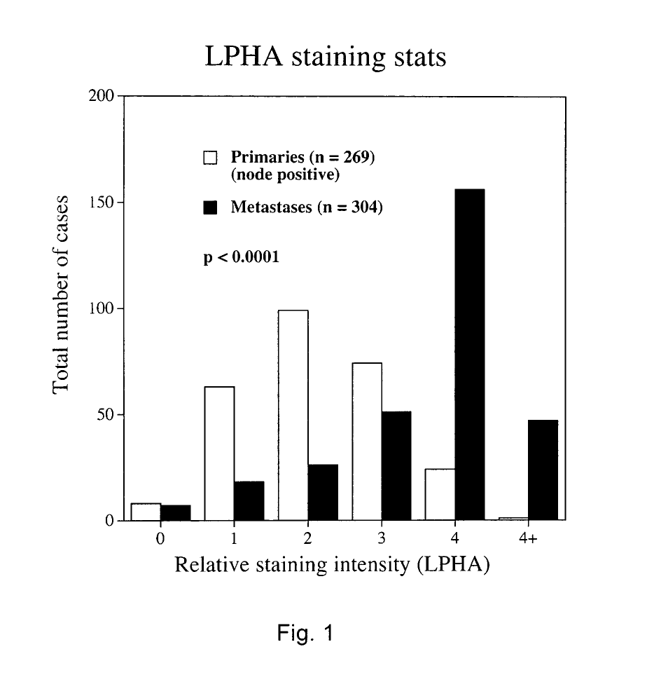

In patient-matched tumor microarrays stained side-by-side with LPHA and hematoxylin, it can be seen that overall LPHA staining intensity is higher in the nodal metastases compared to the primary tumors. Tumor cells in each section were scored for LPHA staining intensity with relative scores of 0-4. The results are shown graphically in FIG. 1, demonstrating a highly significant (p<0.0001), elevation in staining intensity by metastases compared to ‘node-positive’ primary tumors.

These studies were expanded to include ‘node negative’ primaries. In toto, LPHA staining intensity scoring of ‘node-negative’ primary breast carcinomas averaged approximately 1 (n=˜200 tumors); that of ‘node-positive’ primaries, approximately 2 (n=˜500 tumors); and that of carcinoma-positive nodes approximately 4 (n=˜300 tumors). Thus, β1,6-branched N-glycans increased with tumor progression, indicative of a role for these structures in metastasis.

These results also indicated that β1,6-branched oligosaccharides are a common feature of carcinomas of the breast, particularly metastatic tumors. In preliminary studies, similar results were found with a smaller number of metastatic melanomas (n=13), renal cell carcinomas (n=3), and Hodgkin's lymphomas (n=13).

Therefore, it is found that such oligosaccharides and structures associated with them, including polylactoseamines and fucosylated modifications such as Lewis^{x} structures, are targets for diagnostic tests and/or therapeutic intervention, particularly with certain oligosaccharide-targeting bacteria and viruses, lectins, liposomes, antibodies, and the like. Further, certain β1,6-branched oligosaccharide-containing glycoproteins, glycolipids, or glycosaminoglycans expressed by cancer cells, particularly metastatic cancer cells, are also targets for diagnostic tests and/or therapeutic intervention by said agents. Such glycoproteins include lysosome-associated proteins 1 and 2, β1 integrins, CD63, and MAC-1.

### EXAMPLE 2

Bordetellae as metastasis-targeting vectors. The Bordetellae, including Bordetella pertussis, Bordetella parapertussis, and Bordetella bronchiseptica are closely related gram-negative bacterial subspecies that cause respiratory tract infections in humans and other mammals. For example, in its normal life cycle, Bordetella pertussis infects the human airways by attaching to specific oligosaccharides and proteins on respiratory tract cells, such as ciliated epithelia and macrophages (Tuomanen E. Subversion of leukocyte adhesion systems by respiratory pathogens. ASM News 59: 292-296, 1992.). This is accomplished through ‘adhesins’, bacterial proteins that attach to the mammalian cell surface oligosaccharides and proteins via high affinity (‘lock and key’) binding mechanisms (Saukkonen K, Burnette W N, Mar V L, Masure H R, Tuomanen E I. Pertussis toxin has eukaryotic-like carbohydrate recognition domains. Proc. Natl. Acad. Sci. USA 89:118-122, 1992.). These same, or highly similar, oligosaccharides and proteins of the respiratory tract cells are also present in metastatic human tumors, and it is shown herein that Bordetella pertussis use these targets for invasion of human cancer cells in vitro.

Because the Bordetellae possessed specific mechanisms for attachment to cancer cells, e.g. to specific oligosaccharides and proteins aberrently expressed on cancer cells, particularly on metastatic cancer cells, and for additional reasons described below, the Bordetellae are useful as diagnostic aids and tools, diagnostic imaging agents, and as anticancer vectors, particularly for metastases, in humans and other mammals.

Certain non-Bordetellae bacterial species and subspecies with similar inherent specificities for aberrant oligosaccharides and corresponding glycoconjugated proteins on cancer cells are also useful as said anticancer vectors and as agents and tools for diagnosis, e.g., diagnostic imaging. Further, through genetic engineering techniques, appropriate organisms for targeting of cells may be constructed, for example expressing the Bordetella adhesin in other modified species. Likewise, Bordetella may be genetically modified as appropriate to more selectively target certain cells and/or to have a particular effect on these cells or their surrounding tissues.

### EXAMPLE 3

Discrimination between neoplastic and normal human cells by Bordetella pertussis. Human metastatic melanoma cells (Skmel-23/C22) were compared side-by-side to normal human melanocytes and normal human fibroblasts as hosts for invasion of B. pertussis strain 536 (ATCC 10380). The bacteria invaded melanoma cells 20-30 times more than they invaded normal melanocytes and fibroblasts during the same 30 minute time period. Thus, B. pertussis is a tumor-specific vector, associated with its ability to discriminate between cancerous and normal cells, reducing potential unwanted side-effects to normal cells during therapy therewith. B. pertussis is further advantageous in diagnostic imaging due to its ability to discriminate between cancerous and normal cells, thus reducing background false signals from normal cells.

TABLE 1
Comparative invasion of normal and neoplastic human
cells in culture by B. pertussis strain 536.

| | cfu/well ± S.D. | Relative Invasion |
|---|---|---|
| Human cells | invading bacteria | (% of melanoma cells) |
| Skmel-23 human melanoma | 6.9 ± 1.8 × 10⁴ | 100 |
| Normal human melanocytes | 3.6 ± 0.7 × 10³ | 5 |
| Normal human fibroblasts | 1.7 ± 0.6 × 10³ | 3 |

Invasion assays were carried out for 30 minutes, followed with polymixin B.

Bordetella pertussis was cultured for 48-72 h on Bordet-Gengou agar plates (Remel, Inc.) in a 37° C. incubator. Prior to exposure of bacteria to mammalian cells (15-30 minutes), the bacteria were loop-transferred to Luria-Bertani (LB) liquid growth medium and adjusted to a concentration of 10⁹ cfu/ml (OD₆₀₀=0.5). Human Skmel-23/C22 metastatic melanoma cells, normal human melanocytes, or normal human fibroblasts were inoculated into Corning 12 well tissue culture plates (2-4×10⁴ cells/well) in antibiotic-free DMEM growth medium supplemented with 10% fetal bovine serum, and placed in a gassed, humidified incubator, at 37° C. After 24 h, melanoma cells were fed with 1 ml fresh medium, and 15-20 h later, Bordetella pertussis strain 536 was added as described below. Potential inhibitors of Bordetella attachment and invasion of melanoma cells were added immediately before, or up to 2 h before addition of bacteria, as noted. The assay for Bordetella invasion was as follows. Bacteria (0.1 ml in LB liquid medium) were added directly to the melanoma cell culture media to achieve 10⁶-10⁸ cfu/well, depending upon the experiment. The 12 well plates were then incubated at 37° C. After 30 minutes, the medium was replaced with fresh DMEM/FBS containing polymixin B (100 μg/ml), and incubation was continued for an additional 60 minutes. (Since polymixin B is unable to penetrate mammalian cells, non-invading bacteria, outside the cancer cells, were killed by this procedure, while invading bacteria, within the cancer cells, were not.) The polymixin B-containing medium was replaced with Ca⁺⁺/Mg⁺⁺-free saline containing trypsin (0.25% wt/vol) and EDTA (1 mM) and the plates were incubated an additional 10 minutes, 37° C., to harvest the melanoma cells. Melanoma cells were then plated onto Bordet-Gengou agar plates in serial dilutions, incubated at 37° for 4d, and Bordetella pertussis were quantitated by colony counts as ‘colony forming units’ (cfu). Glycosidase F (Peptide N-glycosidase; PNGase F; EC 3.5.1.52) was from Sigma-Aldrich Co.; lectin LPHA (leucocytic phytohemagglutinin from phaseola vuigaris) was from Vector Laboratories, Inc.; anti-CD11B (rat anti-mouse monoclonal antibody CBL 1313 with anti-human reactivity), was from Cymbus Biotchnology LTD; anti-CD15 (mouse anti-human monoclonal antibody clone C3D-1) was from Dako, Inc.) lectin TGP (from tetragonolobus purpureas), RGD (Arg-Gly-Asp) and L-fucose were from Sigma-Aldrich, Co.

### EXAMPLE 4

Visualization of Fluorescence-Labelled B. Pertussis During Attachment and Invasion of Melanoma Cells.

Fluorescent-labelled (FITC) Bordetella pertussis can be seen through a fluorescent microscope attaching to, and/or invading Skmel-23/C22 human metastatic melanoma cells in culture. Invasion procedures with polymixin B were as described above, only using FITC-labelled bacteria, and with extensive saline rinses prior to photography. Comparing the fluorescence field image with a fluorescent plus bright field optic photograph, reveals that the bacteria are within, or attached to the melanoma cells. These results provide proof of attachment to, and/or invasion of B. pertussis into human cancer cells.

Structural requirements on cancer cells for attachment and invasion of B. pertussis. Quantitative invasion assays were carried out with various additives to investigate structural requirements on human melanoma cells for attachment and invasion of B. pertussis. All additives listed in Table 1 below inhibited invasion of Bordetella pertussis as noted. The implied targets revealed by the inhibitors are listed in the right-hand column.

TABLE 2
Use of inhibitors to deduce structural requirements
for Bordetella attachment and invasion into human
melanoma cell line Skmel-23/C22.

| | Bordetella (cfu/well) | |
|---|---|---|
| Additive | (% Control +/− S.D.) | Implied target |
| H₂O (control) | 100 ± 11 | n.a. |
| Glycosidase F (1 unit/ml) | 47 ± 5 | N-glycans |
| Lectin LPHA (50 μg/ml) | 68 ± 21 | β1,6-branched |
| | | N-glycans |
| Anti-CD15 (1:20) | 43 ± 15 | Lewis^{x}, CD15 |
| Lectin TGP (50 μg/ml) | 44 ± 11 | Fuc(α1-2)Galβ1- |
| | | 3GlcNac |
| L-fucose (0.2% wt/vol) | 52 ± 14 | fucosylated structures |
| Anti-CD11b (1:20) | 29 ± 13 | CD11b, MAC-1 |
| RGD (0.8 mg/ml) | 50 ± 5 | Arg-Gly-Asp |
| | | tripeptide sequence |

Melanoma cells were incubated with additives 2 h prior to addition of bacteria, except for L-fucose, which was added 5 min. prior to bacteria. Invasion assays were carried out for 30 minutes followed by addition of polymixin B as described above.

Therefore, B. pertussis attachment and invasion involved melanoma cell N-glycans, at least some of which were β1,6-branched N-glycans, and at least some of which contained fucosylated structures such as Lewis^{x}. Protein/peptide attachment sites on melanoma cells for B. pertussis included MAC-1 or MAC-1-like sequences, and Arg-Gly-Asp tripeptide sequences. Thus, it is apparent that Bodetella uses these structures during the process of invading cancer cells. Further, according to the present invention, these structures are thus targets for therapeutic intervention with Bordetella anticancer vectors. Likewise, other organisms which naturally target these cell markers, or which are engineered to target these markers, may also be used in accordance with the present invention. It is further a part of this invention that these structures are thus targets for diagnosis, e.g., diagnostic imaging with Bordetella vectors.

### EXAMPLE 5

Bordetella Pertussis Toxicity Toward Cancer Cells.

Exposure of cancer cells in culture to Bordetella pertussis and/or to substances released by Bordetella pertussis, caused rapid morphological changes, cytotoxicity, and death of human cancer cells in culture. Cancer cell cytotoxicity has also been shown in vivo in mice (discussed in more detail below), and it is therefore an aspect of the present invention to use Bordetella pertussis as a therapy for human or mammalian cancer.

Skmel-23/C22 human melanoma cells were cultured 15 h with or without the addition of B. pertussis to the culture medium. B. pertussis caused bizarre dendrite extensions, followed by disintegration and death of the cells.

Thus in addition to use of Bordetella pertussis as a vector, the organism can be used to deliver inherently-produced anticancer toxins to tumors. Also, Bordetella pertussis can be genetically-engineered to produce additional agents with anticancer activities, such as toxins, prodrug converting enzymes, cytokines, and the like. See, e.g., U.S. Pat. No. 6,190,657, expressly incorporated herein by reference.

### EXAMPLE 6

Histidine-Mediated Toxicity Of Bordetella Pertussis Toward Cancer Cells.

During investigations of B. pertussis invasion into cancer cells in vitro, a potent cytotoxicity was exhibited by wild type bacteria strain Tohama I (ATCC BAA-589, NCTC 13251) toward a variety of cancer types. A derivative of Tohama 1, strain, 536, showed similar toxicity, but only in the presence of the amino acid histidine. Cancer cells tested were human carcinomas of the breast, lung, and kidney, and melanoma. Toxicity of B. pertussis strain 536, but not of Tohama I, was dependent on the simultaneous addition of rich nutrient broth such as Luria-Bertani bacterial growth medium, or amino acid-rich broths such as tryptone or casamino acids. In the presence of these mixtures, but not in the presence of an equivalent volume of physiologic saline, cancer cells challenged with B. pertussis 536 showed signs of acute stress within 6 hours, and massive lysis (>90% of cells) by 24 hours. The principal active ingredient in these broths was the amino acid histidine. In the absence of histidine, the bacteria invaded tumor cells and colonized them for at least 7 days, but exhibited little or no signs of toxicity toward the cancer cells. Of 18 amino acids tested, only histidine induced a Bordetella-mediated cytotoxic/lytic effect. Histidine alone showed no toxicity.

Histidine, histidine analogs, or other amino acids and related compounds, may therefore advantageously be used for activating B. pertussis-mediated cytotoxicity in tumors. Bordetella-mediated cytotoxicity induced within the tumor, can be regulated through systemic or oral administration of histidine or analogs to cancer patients with Bordetella-colonized tumors.

Through such administration, toxicity may be regulated directly within the tumor, with little toxicity to normal tissues. Sustained or pulsed regulation of toxicity could be achieved through the timing of administration.

TABLE 3
Histidine-mediated toxicity of B. pertussis strain 536
toward SKMel-23 human melanoma cells in culture

| | melanoma cells/well (24 h post-treatment) | |
|---|---|---|
| B. pertussis | Saline(% | L-histidine(% |
| Strain | untreated cultures) | untreated cultures) |
| none | 1.6 ± .09 × 10⁵ (100%) | 1.7 ± .07 × 10⁵ (106%) |
| Tohama I | 1.5 ± 0.5 × 10⁴ (9%) | 1.4 ± 0.2 × 10⁴ (8%) |
| (wild type) | | |
| 536 | 1.0 ± .04 × 10⁵ (63%) | 1.4 ± 0.2 × 10⁴ (8%) |

### EXAMPLE 7

Targeting Of Human Lung Carcinoma A549 Growing In Nu/Nu Mice

Bordetella pertussis successfully targeted and colonized human lung carcinoma implanted in nu/nu mice (Table X). It is thus suggested that B. pertussis, when introduced into the bloodstream of a cancer patient, would similarly target metastatic tumors. The attachment and invasion capabilities of B. pertussis demonstrated above provide novel and highly selective mechanisms for targeting human tumors. Thus, as part of this invention, Bordetella pertussis is useful for targeting human tumors for the purposes of destroying tumor cells or otherwise inhibiting tumor growth.

TABLE 4
B. pertussis targeting of human lung carcinoma A549 growing
s.c. in nude (nu/nu) mice following tail vein injections of bacteria.

| | Days post | tumor | |
|---|---|---|---|
| Mouse # | bacteria | wt | cfu/g tumor |
| 1 | 2 | 0.5 g | 4 × 10⁶ |
| 2 | 2 | 0.5 g | 5 × 10⁴ |
| 3 | 2 | 0.5 g | 8 × 10⁶ |
| 4 | 2 | 0.5 g | none detected |
| 5 | 7 | 0.7 g | 3 × 10⁸ |
| 6 | 21 | 0.2 g | 5 × 10⁶ |

10⁸ cfu B. pertussis strain 536 were injected i.v. At the times indicated mice were sacrificed through approved euthanasia techniques. Tumors were removed, weighed, and homogenized in 3 vol LB broth/g tumor. Bacteria were quantitated by serial dilutions of the homogenates onto Bordet-Gengou agar plates, incubating at 37° C., and counting B. pertussis colonies 4-5 days later.

### EXAMPLE 8

Immunotherapy with Bordetella Pertussis.

Most individuals have been vaccinated, and/or carry natural immunity toward B. pertussis, predicting that colonization of tumors by this strain would elicit a delayed but strong intratumoral immune response toward both bacteria and cancer cells. Thus, due to the inherent immunogenicity of Bordetella pertussis, it would be useful in immunotherapy against tumors colonized by the bacteria, particularly metastatic tumors. It is further noted that Bordetella pertussis additionally genetically engineered to express non-Bordetella immunogens or cytokines, capable of eliciting anti-tumor immune responses, are also useful as immunotherapeutic agents in cancer treatment. Likewise, the organisms may be labeled with an NMR, radioactive, fluorescent, or other label, such that their localization in the body may be determined after administration. Where a strong local immunologic reaction takes place, this may also be located or visualized in known manner, to determine the target position. Likewise, immunotherapies may be administered, for example prior to or in conjunction with the administration of the targeting agent, to enhance the local response.

With localization of the target, other targeted therapy, such as radiation therapy, photodynamic therapy, chemotherapy, or the like, may also be applied. Therefore, according to this embodiment, it is not necessary that the targeting organism or composition itself be cytotoxic or directly generate a cytotoxic response, rather, that it target specifically and reliably, with therapy applied as a separate measure.

### EXAMPLE 9

Treatment of Aerobic Regions of Tumors.

An important property of the Bordellae is that they are aerobic bacteria, and in that regard would be metabolically active in vascularized aerobic regions of tumors, notably the areas of highest tumor growth rate. Thus, as part of this invention Bordetellae are useful for colonizing small tumors wherein there is little or no necrosis, and most of the tumor is vascularized and aerobic. Thus, the invention does not depend on the presence of large tumors. Likewise, since the targeting is at a cellular level, rather than a tissue level, even small clusters of cells may be affected by this treatment.

### EXAMPLE 10

Combination Therapies.

Bordetellae and certain additional non-Bordetellae, oligosaccharide-targeting bacteria can be used alone or in combination with other bacterial vectors with complementary anticancer capabilities. Bordetellae could also be used in combination with other therapeutic agents such as X-rays, chemotherapeutic drugs, and biotherapeutic agents.

### EXAMPLE 11

Safety Testing in Mice.

LD₅₀ studies in mice demonstrated that injection of wild type B. pertussis into the bloodstream had no noticeable toxic side effects to the animals, even after 3 repeated injections of the highest feasible doses (10⁹ per animal). Thus, in mice, B. pertussis did not elicit septic shock, even when injected at levels exceeding by more than 100 fold the levels known to cause septic shock and death following similar injection of E. coli or Salmonella. It is thus apparent that, according to the present invention, wild type Bordetella pertussis can be used as an anticancer vector, without further attenuation, to avoid triggering septic shock. This therefore minimizes the risk of environmental release of a modified pathogenic organism.

However, in certain circumstances, further attenuation may be necessary and desirable, and thus, according to the present invention, Bordetella pertussis anticancer vectors may be provided which are attenuated in virulence.

The present invention therefore provides for exploitation of aberrant oligosaccharides and the corresponding glycoconjugated proteins and lipids on cancer cells, for targeting and therapy of tumors, particularly metastatic tumors, by certain oligosaccharide-targeting bacteria and viruses, lectins, liposomes, antibodies, pharmaceuticals, macromolecules, and the like. The present invention also supports the use of agents and vectors which target these aberrant oligosaccharides as diagnostic tools. The diagnosis may include subjecting biopsy samples to oligosaccharide-specific agents, in vivo administration of oligosaccharide-specific agents, blood tests, or the like. The present invention therefore encompasses specific organisms as vectors, pharmaceutical and diagnostic agents which may be administered orally, intravenously, transmucosally, or through other portals of entry, methods of treatment and/or diagnosis employing these agents and vectors, apparatus designed to administer the agents or vectors, and apparatus to image or diagnose pathology, and pharmaceuticals intended to control side effects of the diagnosis or treatment, for example antibiotics.

### EXAMPLE 12

Human Lung Carcinoma A549: Tumor Growth Suppression and Regression In Nu/Nu Mice Following Treatment with B. Pertussis.

Human lung carcinoma A549 was implanted subcutaneously into ‘nude’ (nu/nu) mice. These mice are genetically immuno-suppressed, and are thus permissive hosts for human tissue. Eight weeks after implantation of tumor cells, solid tumors averaging 200-400 mg could be palpated in 24 of the animals. These animals were divided into 2 groups 1) control: saline-injected (n=8 mice); 2) experimental: Bordetella pertussis-injected (10⁹ cfu bacteria per mouse) (n=16 mice). Mice were injected with a 1:1 mixture of B. pertussis wild type strain Tohama I, and mutant strain Bp 536. Each mouse was injected intraperitoneally and intratumorally. Bacterial or saline control injections were repeated every 1-2 wks. Individual mice were marked, and the specific tumor growth in each mouse was followed. Below is a summary 40 days after the first bacterial injections.

Growth of Tumors

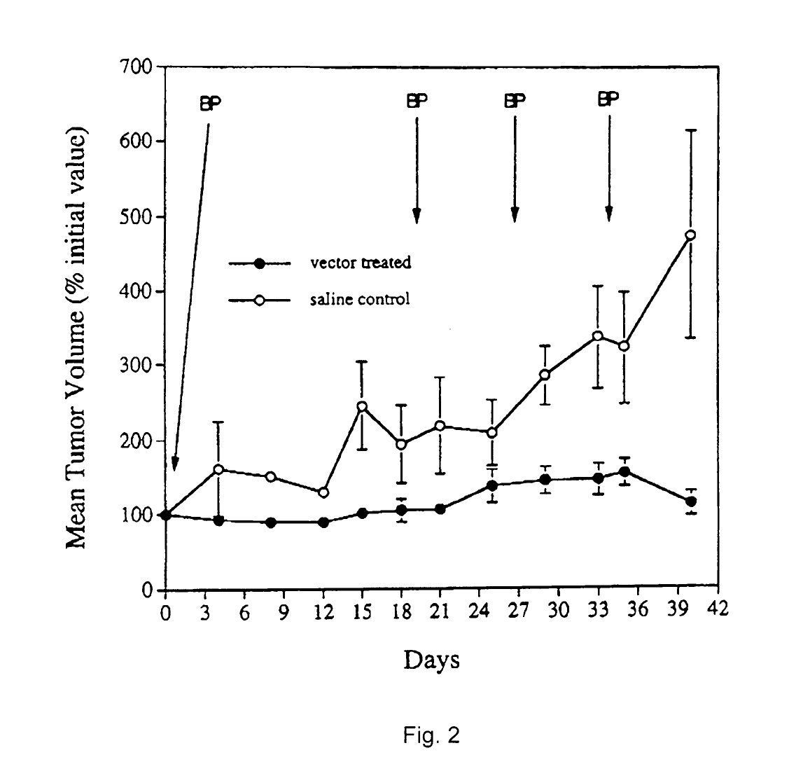

The results are graphically shown in FIG. 2.

1. Control Mice (Open Circles):

Of the 8 saline-injected controls, three mice died from the tumor. Of the remaining animals, each individual tumor steadily increased in mass over 40 days, such that there has been a mean 5-fold increase in tumor mass in the control animals. The control tumors were well-vascularized with no ulceration or scarring.

2. Bacteria-Treated Mice (Closed Circles)

Of the 16 bacteria-injected animals, although there was some initial tumor growth each individual tumor eventually showed decreases in tumor mass, such that at 40 days after initiation of treatment the mean tumor size of the population was somewhat less than the starting size. In three mice, the tumor regressed to no measureable tumor mass at all. In most of the bacteria-treated mice, there was ulceration of tumors and build-up of scar tissue accompanying regressions.

3. Safety: The mice received 4 doses of bacteria over 40 days. Each dose was of about 10⁹ colony forming units (cfu) per mouse, with no noticeable side-effects. Thus these dosages of vectors showed little or no toxicity to mice.

### EXAMPLE 13

Human tumor DNA was analyzed for evidence of hybrid cells, in a child who developed metastatic renal cell carcinoma, subsequent to an allogeneic bone marrow transplant. A metastasis was searched for the donor's A blood group allele within the O/O recipient's tumor cells. Tumor cell clusters in diverse regions of the metastasis were microdissected from fixed tissue sections. Of the 21 DNA samples tested, 16 yielded PCR products, and all 16 contained the donor A allele. The most probable explanation is that the metastasis contained donor-recipient hybrids throughout. Tumor cells also stained for myeloid-type oligosaccharides, a trait of experimental macrophage-tumor cell fusion hybrids. The findings suggest tumor-hematopoietic cell hybridization as a cause of metastatic progression in this patient.

Fusion between bone marrow-derived stem cells and liver cells has recently been implicated as a mechanism for liver regeneration in mice (1-2). These results make it reasonable to reconsider the hypothesis that metastases arise when a tumor-infiltrating macrophage, which possesses many of the properties of a metastatic cell, fuses to a tumor cell. The concept that leucocyte-tumor cell hybridization may be a causal event in malignancy was first put forth nearly a century ago (3-5). Since then, there have been numerous reports in animal tumor models of spontaneous fusion hybrids between implanted tumor cells and normal tumor-infiltrating cells of the host (6-9). Studies on fusion hybrids in animals are possible because heterologous genetic markers were employed to distinguish parental genotypes. To explore the possibility of hybridization in human cancer, we examined a formalin-fixed, paraffin-embedded metastasis of a renal cell carcinoma in a lymph node of a 5 year old boy who, 8 months prior to detection of metastasis, had received a bone marrow transplant (BMT) from his HLA-identical sibling (a brother). The patient's ABO typing was O+ and that of the donor, A+. Tumor cells were isolated by laser microdissection microscopy (10). DNA was extracted, and using primer sets designed to amplify A and O blood group alleles, specific amplified fragments were identified by agarose gel electrophoresis, and in some cases by sequencing of bands isolated from the gels. In addition, through lectin histochemistry with LPHA (leukocytic phytohemagluttinin, phaseola vulgaris), tumor sections were stained for β1,6-branched oligosaccharides. These complex sugars, normally expressed by myeoloid cells such as macrophages and granulocytes, are also a prominent feature of experimental macrophage-melanoma hybrids (11), and co-expression of these sugars along with coarse cytoplasmic vesicles has recently been shown to be a common and pervasive phenotype for a wide variety of human solid tumors, particularly metastases (12). From both the genetic and histopathologic studies, the data appear to support the concept that the metastatic tumor described herein was composed predominantly of donor-recipient fusion hybrids.

The results indicate that donor DNA, as represented by the A allele, was ubiquitous in tumor cells throughout the metastasis. It seems most likely that this was due to fusion hybridization between donor BMT cell(s) and recipient tumor cell(s). Since the metastasis contained donor DNA throughout, it would also seem likely that the hybridization event(s) occurred early in the generation of the metastasis, probably in the primary tumor.

The nature of the donor cell fusion partner would be of great interest. It was earlier proposed that metastatic hybrids might be formed through aberrant phagocytosis of apoptotic tumor cells by tumor-infiltrating phagocytes such as macrophages (6-8), and indeed horizontal transfer of genetic information during phagocytosis of apoptotic bodies has been observed in culture (16-19). Further, it was recently shown in mice that bone marrow-derived stem cells appear to hybridize with pre-existing hepatocytes during stem cell-mediated liver regeneration (1-2). Since bone marrow-derived stem cells and all blood lineages are presumably replaced with the donor cells after BMT, numerous hematopoietic cell types are thus potential candidates as fusion partners with the primary carcinoma. LPHA lectin-histochemistry of this tumor revealed wide-spread expression of β1,6-branched oligosaccharides and coarse vesicles—normal traits of myeloid cells such as macrophages and granulocytes (20). This phenotype is also a prominent trait in experimental macrophage-melanoma hybrids, and is a common, pervasive phenotype in human cancers, particularly in metastases (12). In a recent case study of another renal carcinoma patient, only a minor population of the primary tumor consisted of LPHA-positive, vesicular tumor cells, whereas a vast majority of metastatic cells in the lung and spinal cord had this phenotype (112). This observation suggests that the LPHA-positive cells in the primary tumor were those with high metastatic potential.

A general, prevailing view of metastasis is that tumor progression results from genetic variability within the original clone, allowing for sequential selection of more aggressive sublines' (21). Much recent work has focused on delineation of gene expression signatures associated with metastatic progression (22-27). A tumor hybridization model would address the underlying basis of such signatures, as well as the initiating events in metastatic transformation. Notably, hybrid tumor cells would tend to be aneuploid, a trait highly associated with metastasis (3-6, 28). A hybrid phenotype would depend upon the number and nature of parental genes incorporated into the hybrid genome, and, in theory, would be determined by dominant-recessive relationships between the different developmental lineages of the parental fusion partners. Whether the hybrid constituted a minor or major component of the tumor population would depend on the timing of fusion hybridization during expansion of the primary tumor, as well as the cell-cycle length of the hybrid compared to other tumor cells.
1. Wang, X. et al. Cell fusion is the principle source of bone-marrow-derived hepatocytes. Nature 422, 897-901 (2003).
2. Vassilopoulos, G., Wang, P-. & Russell, D. W. Transplanted bone marrow regenerates liver by cell fusion. Nature 422, 901-904 (2003).
3. Aichel, O., Eine neue Hypotheses über Ursachen und Wesen bosartiger Geschwülste. J. F. Lehmann. München (1908).
4. Aichel O. Uber Zellverschmelzung mit qualitative abnormer chromosomenverteilung. In: Roux W, ed. “Vorträge und Aufsätze über Entvickelungsmechanik Der Organismen”. Leipzig, Germany: Wilhelm Engelmann, pp. 1-115 (1911).
5. Boveri, T. The Origin of Malignant Tumors. Williams and Wilkins, Co., Waverly Press, Baltimore. pp 1-119 (1929).
6. Pawelek, J. M. Tumor Cell Hybridization and Metastasis Revisited. Melanoma Res. 10, 507-514 (2000).

7. Rachkovsky et al. Enhanced metastatic potential of melanoma x macrophage fusion hybrids. Clin Exp Metastasis 16, 299-312 (1998).
8. Chakraborty, et al. A spontaneous murine melanoma lung metastasis comprised of host x tumor hybrids. Cancer Research 60, 2512-2519 (2000).
9. Duelli, D. & Lazebnik, Y. Cell fusion: A hidden enemy? Cancer Cell 3, 445-448 (2003).
10. Persson, A. H., Backvall, H., Ponten, F., Uhlen, M. & Lundeberg, J. Single cell gene mutation analysis using laser-assisted microdissection of tissue sections. Methods 13 Enzymol. 356, 334-343 (2002).
11. Chakraborty, A. K. et al. Macrophage fusion up-regulates N-acetyl-glucosaminyltransferase β1_{_{—}}branching, and metastasis in Cloudman S91 mouse melanoma cells. Cell Growth and Differentiation
12, 623-630 (2001). 12. Handerson, T. & Pawelek, J. β1,6-branched oligosaccharides and coarse vesicles: A common and pervasive phenotype in melanoma and other human cancers. Cancer Research. In press (September 2003).
13. Perkins, J. L, Neglia, J. P., Ramsay, N. K. C. & Davies, S. M. Successful bone marrow transplantation for severe aplastic anemia following orthotopic liver transplantation: long-term follow-up and outcome. Bone Marrow Transplant 2, 523-526 (2001).
14. Socie G. et al. New malignant diseases after allogeneic marrow transplantation for childhood acute leukemia. J. Clin. Oncol. 18, 348-357 (2000).
15. Ades, L., Guardiola, P. & Socie, G. Second malignancies after allogeneic hematopoietic stem cell transplantation: new insight and current problems. Blood Rev. 16,135-146 (2002).
16. de la Taille A., Chen M., Burchardt M., Chopin D. K., Buttyan R. Apoptotic Conversion: Evidence for Exchange of Genetic Information between Prostate Cancer Cells Mediated by Apoptosis. Cancer Research 59, 5461-5463 (1999).
17. Holmgren, L. et al. Horizontal transfer of DNA by the uptake of apoptotic bodies. Blood 93, 3956-3963 (1999).
18. Bergsmedh, A. et al. Horizontal transfer of oncogenes by uptake of apoptotic bodies. Proc. Natl. Acad. Sci. USA 98, 6407-6411 (2001). 14
19. Bergsmedh, A., Szeles, A., Spetz, A. L. & Holmgren, L. Loss of the p21 (Cip1/Waf1) cyclin kinase inhibitor results in propagation of horizontally transferred DNA. Cancer Res. 62, 575-579 (2002).
20. Fukuda M., Spooncer E., Oates J. E., Dell A, & Klock J. C. Structure of sialylated fucosyl lactosaminoglycan isolated from human granulocytes. J. Biol. Chem. 25, 10925-10935 (1984).
21. Nowell P. C. The clonal evolution of tumor cell populations. Science 194, 23-28 (1976).
22. van de Vijver, M. J. et al. A gene-expression signature as a predictor of survival in breast cancer. N Engl J. Med. 347, 1999-2009 (2002).

23. Ramaswamy, S. Ross, K. N., Lander. E. S. & Golub, T. R. A molecular signature of metastasis in primary solid tumors. Nature Genetics 33, 49-54 (2003).
24. Hunter, K., Welch, D. R. & Liu, E. T. Genetic background is an important determinant of metastatic potential. Nature Genetics 34, 23-24 (2003).
25. Fidler, I. J. & Kripke, M. L. Genomic analysis of primary tumors does not address the prevalence of metastatic cells in the population. Nature Genetics 34, 23 (2003).
26. Ramaswamy, S., Ross, K. N., Lander, E. S. & Golub, T. R. Reply to “Genomic analysis of primary tumors does not address the prevalence of metastatic cells in the population” and “Genetic background is an important determinant of metastatic potential.” Nature Genetics 34, 25 (2003).
27. Couzin, J. Tracing the steps of metastasis, cancer's menacing ballet. Science 299, 1002-1006 (2003). 15
28. Li, R., Sonik, A., Stindl, R., Rasnick, D. O. & Duesberg, P. Aneuploidy vs. gene mutation hypothesis of cancer: Recent study claims mutation but is found to support aneuploidy. Proc. Nat. Acad. Sci. USA 97, 3236-3241 (2000).
29. Yamamoto, F. et al. Cloning and characterization of DNA complementary to human UDP-GalNAc: Fucα1->2galα1->3GalNAc Transferase (Histoblood Group A Transferase) mRNA. J. Biol. Chem 265, 1146-1151 (1990).
30. Lee, J. C-I. & Chang, J-G. ABO genotyping by polymerase chain reaction. J. Forensic Sci. 37, 1269-1275 (1992).
31. Subrahmaniam, Y. V. B. K., Baskaran, N., Newburger, P. E. & Weissman, S. A modified method for the display of 3′-end restriction fragments of cDNAs: Molecular profiling of gene expresion in neutrophils. Meth. Enzymol. 303, 272-297 (1999).

### EXAMPLE 14

Diagnostic Test for Macrophage-Tumor Derived Hybrid Cells

The existence of hybrid cells may be determined by a dual label technique including a combination of tests for lectins, indicative of myeloid-type oligosaccharides, and tumor-specific markers. This test need not be of the same specimen, and for example, may be of adjacent sections of a biopsy, or the like. Of course, the same specimen could be labeled with different indicators, with the presence of both indicators representing a hybrid. As noted above, care should be exercised to separate normal myeloid cells from the putative hybrids, either physically prior to determination, or by observing cellular boundaries during examination.

Lectins (for Myeloid Type Oligosaccharides):

a) lectin LPHA (leukocytic phytohemagglutinin) (Cummings, R. D., and Kornfeld, S. Characterization of the structural determinants required for the high affinity interaction of asparagine-linked oligosaccharides with immobilized phaseolus vulgaris leukoagglutinating and phytoagglutinating lectins. J. Biol. Chem. 257: 11230-11234, 1982; Fernandes B., Sagman U., Auger M., Demetrio M., Dennis J. W. β1,6-branched oligosaccharides as a marker of tumor progression in human breast and colon neoplasia. Cancer Res. 51: 718-723, (1991).
b) peanut agglutinin lectin (Tuomanen et al. Receptor analogs and monoclonal antibodies that inhibit adherence of Bordetella pertussis to human ciliated respiratory epithelial cells. J. Exp Med 168: 267-277, (1988)
c) tetragonologus purpureas lectin (Tuomanen et al. Receptor analogs and monoclonal antibodies that inhibit adherence of Bordetella pertussis to human ciliated respiratory epithelial cells. J. Exp Med 168: 267-277, (1988)
   Tumor Specific Markers:
melanoma (S100 antibody against protein gp100)
carcinomas (lung, breast, colon, etc) (antibody to cytokeratin)
   Method for Diagnosis of Metastatic Cells in a Primary Tumor:

Using biotinylated or fluorescent-labelled lectins as diagnostic tools, combine any or all of the lectins a-c (or additional appropriate lectins) with tumor-specific antibodies. Staining need not be in combination, as individual stains can be applied to sequential sections of the tumor.

The invention claimed and described herein is not to be limited in scope by the specific embodiments herein disclosed since these embodiments are intended as illustrations of several aspects of the invention. Indeed, various modifications of the invention in addition to those shown and described herein will become apparent to those skilled in the art from the foregoing description. Such modifications are also intended to fall within the scope of the appended claims.

1. A method of detecting or treating mammalian tumor cells, comprising administering an effective amount of a β1,6-branched oligosaccharide specific binding agent to the mammal, wherein the β1,6-branched oligosaccharide specific binding agent is associated with an imaging agent or cytotoxic process.

2. The method of claim 1, wherein the tumor cells are derived from a cell type selected from the group consisting of a metastatic carcinoma, metastatic melanoma, brain tumor, lymphoma, and myelogenous leukemia.

3. The method of claim 1, wherein the tumor cells are derived from a cell type selected from the group consisting of breast, kidney, melanocyte, and lymphocyte.

4. The method of claim 1, wherein the β1,6-branched oligosaccharide binding agent specifically binds to oligosaccharides characteristic of myeloid cell lines.

5. The method of claim 1, wherein the β1,6-branched oligosaccharide binding agent specifically binds to oligosaccharides characteristic of human macrophages

6. The method of claim 1, wherein the binding agent comprises a bacterium.

7. The method of claim 6, further comprising the step of administering an antibiotic to the mammal after administering the bacterium.

8. The method of claim 1, wherein the binding agent comprises a bacterium of genus Bordetella.

9. The method of claim 1, wherein the binding agent comprises a bacterium expressing an adhesin corresponding to the adhesin of genus Bordetella.

10. The method of claim 1, wherein the binding agent comprises Bordetella pertussis.

11. The method of claim 1, wherein the binding agent comprises attenuated Bordetella pertussis.

12. The method of claim 1, wherein the binding agent comprises an organism selected from the group consisting of Bordetella pertussis, Bordetella parapertussis, and Bordetella bronchiseptica.

13. The method of claim 1, wherein the binding agent comprises genetically modified Bordetella pertussis.

14. The method of claim 1, wherein the binding agent comprises genetically modified Bordetella strain expressing a gene product which is imageable.

15. The method of claim 1, wherein the binding agent comprises genetically modified Bordetella strain expressing myoglobin.

16. The method of claim 1, wherein the binding agent comprises an antibody.

17. The method of claim 1, wherein the binding agent is cytotoxic.

18. The method of claim 1, wherein the binding agent initiates an endogenous cytotoxic cascade.

19. The method of claim 1, wherein the binding agent interacts with an exogenous agent to initiate a cytotoxic cascade.

20. The method of claim 1 wherein the β1,6-branched oligosaccharide binding agent specifically binds to a saccharide which is conjugated with a protein, lipid, glycosaminoglycan, or saccharide on the cell.

21. The method to claim 1 wherein the β1,6-branched oligosaccharide specific binding agent comprises a bacteria, virus, lectin, liposome, or antibody, having an affinity for cells having aberrant oligosaccharides, and/or their corresponding aberrant glycoconjugated proteins, lipids, and glycosaminoglycans on metastatic tumors.

22. The method according to claim 19, wherein the β1,6-branched oligosaccharide specific binding agent bears inherent or engineered anticancer toxins, chemicals, or bioactive agents, to destroy cancer cells or otherwise inhibit tumor growth.

23. The method according to claim 19, wherein the β1,6-branched oligosaccharide specific binding agent is associated with an imaging agent which is imagable by a method selected from one or more of the group consisting of magnetic resonance imaging, gamma scintillation, positron emission, and specific fluorescence.

24. The method according to claim 1, further comprising the step of administering an antibiotic to the animal to treat infection of the animal by the bacteria.

25. A method, comprising the steps of:
administering living bacteria to a multicellular organism, the bacteria having an affinity for tissue having a predetermined cell surface oligosaccharide pattern; and
determining the presence of the tissue having the predetermined cell surface oligosaccharide pattern dependent on affinity of the bacteria therefore.

26. The method according to claim 25, further comprising the step of imaging a pattern of affinity of the bacteria for the tissue in vivo.

27. The method according to claim 26, further comprising the step of imaging a pattern of affinity of the bacteria for the tissue in vitro.

28. The method according to claim 26, further comprising the step of administering an antibiotic to the animal to treat infection of the animal by the bacteria.

29. A method of assessing malignancy of a tumor, comprising analyzing a glycosylation pattern of the cells using magnetic resonance spectroscopy.

30. The method according to claim 29, wherein the magnetic resonance spectroscopy produces an image corresponding to a position of a glycosylation pattern.

31. The method according to claim 29, wherein a pharmaceutically acceptable composition is administered to a patient having an affinity for a predetermined glycosylation pattern prior to or simultaneous with conducting magnetic resonance spectroscopy.

32. A pharmaceutical formulation for administration to humans, comprising live Bordetellae.

33. The formulation according to claim 32, wherein the Bordetellae are genetically engineered to produce myoglobin.

34. The formulation according to claim 32, wherein the Bordetellae comprise Bordetella pertussis Tohama I: ATCC BAA-589, NCTC 13251.

35. The formulation according to claim 32, wherein the Bordetellae comprise Bordetella pertussis strain 536: ATCC 10380.

36. A pharmaceutical formulation, comprising a β1,6-branched oligosaccharide specific binding agent conjugated to a cytoxin.

37. The pharmaceutical formulation according to claim 36, wherein the specific binding agent is a lectin.
